(19) United States
(12) Patent Application Publication
(10) Pub. No.: US 2025/0152193 A1
(43) Pub. Date: May 15, 2025

(54) THROMBUS REMOVAL SYSTEMS AND ASSOCIATED METHODS

(71) Applicant: Shifamed Holdings, LLC, Campbell, CA (US)

(72) Inventors: Paul GUNNING, Montara, CA (US); Aadel AL-JADDA, San Carlos, CA (US); Amr SALAHIEH, Saratoga, CA (US); Ich ONG, Campbell, CA (US); Muralidharan SRIVATHSA, Santa Clara, CA (US); Praveen Krishna DALA, San Ramon, CA (US); Tom SAUL, Portland, OR (US)

(21) Appl. No.: 18/835,686

(22) PCT Filed: Feb. 3, 2023
(86) PCT No.: PCT/US2023/062002

§ 371 Date: Aug. 2, 2024

Related U.S. Application Data
(60) Provisional application No. 63/267,530, filed on Feb. 3, 2022.
(60) Provisional application No. 63/269,380, filed on Mar. 15, 2022.
(60) Provisional application No. 63/373,413, filed on Aug. 24, 2022.
(60) Provisional application No. 63/373,427, filed on Aug. 24, 2022.

(51) Int. Cl. A61B 17/221; A61B 17/00; A61B 17/22; A61B 17/32

(52) U.S. Cl. CPC A61B 17/221; A61B 17/00234; A61B 17/32; A61B 2017/00238; A61B 2017/00305; A61B 2017/00353; A61B 2017/00477; A61B 2017/00862; A61B 2017/22079; A61B 2017/2215; A61B 2017/32006; A61B 2217/005; A61B 2217/007

(57) ABSTRACT

The present technology relates to systems and methods for removing a thrombus from a blood vessel of a patient. In some embodiments, the present technology is directed to systems including an elongated catheter having a distal portion configured to be positioned within the blood vessel of the patient, a proximal portion configured to be external to the patient, and a lumen extending therebetween. The system can also include a fluid delivery mechanism coupled with a fluid lumen and configured to apply fluid to at least partially fragment the thrombus.

### CROSS REFERENCE TO RELATED APPLICATIONS

This application claims the benefit of priority to U.S. Application No. 63/267,530, filed Feb. 3, 2022, U.S. Application No. 63/269,380, filed Mar. 15, 2022, U.S. Application No. 63/373,413, filed Aug. 24, 2022, and U.S. Application No. 63/373,427, filed Aug. 24, 2022, each of which are herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

The present technology generally relates to medical devices and, in particular, to systems including aspiration and fluid delivery mechanisms and associated methods for removing a thrombus from a mammalian blood vessel.

### BACKGROUND

Thrombotic material may lead to a blockage in fluid flow within the vasculature of a mammal. Such blockages may occur in varied regions within the body, such as within the pulmonary system, peripheral vasculature, deep vasculature, or brain. Pulmonary embolisms typically arise when a thrombus originating from another part of the body (e.g., a vein in the pelvis or leg) becomes dislodged and travels to the lungs. Anticoagulation therapy is the current standard of care for treating pulmonary embolisms, but may not be effective in some patients.

Additionally, conventional devices for removing thrombotic material may not be capable of navigating the tortuous vascular anatomy, may not be effective in removing thrombotic material, and/or may lack the ability to provide sensor data or other feedback to the clinician during the thrombectomy procedure. Existing thrombectomy devices operate based on simple aspiration which works sufficiently for certain clots but is largely ineffective for difficult, organized clots. Many patients presenting with deep clots in difficult to reach anatomical locations and/or deep vein thrombus (DVT) or PE are left untreated as long as the risk of limb ischemia is low.

In more urgent cases, they are treated with catheter-directed thrombolysis or lytic therapy to break up a clot over the course of many hours or days.

More recently other tools like clot retrievers have been developed to treat DVT and pulmonary embolism (PE). Clot retrievers typically include a structure that is deployed from a distal end of the catheter within the vessel to capture thrombus and then withdrawn back into the distal end of the catheter for thrombus removal. The structure can include stent-like structures, expandable capture baskets, or capture structures that include passive capture features like rakes, barbs, or prongs to engage the clot. These tools are not being widely adopted because of their limited effectiveness, high mortality rates, and additional costs versus aspiration or the standard of care. Additionally, advancing the capture structure distally from the end of the catheter poses additional challenges including limited visualization of the clot relative to the capture device and the risk of damaging vessel walls with the passive capture structures. Other recent developments focus on slicing or macerating the clot, but these mechanisms are designed to reduce the risk of the catheter clogging and do not address the problem of tough, large, organized clots. There remains the need for a device to address these and other problems with existing venous thrombectomy including, but not limited to, a fast, easy-to-use, and effective device for removing a variety of clot morphologies in difficult to reach anatomical locations.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1-1L illustrate various views of a portion of a thrombus removal system including a distal portion of an elongated catheter configured in accordance with an embodiment of the present technology.

FIGS. 2A-2E illustrate plan views of various configurations of irrigation ports and fluid streams of a thrombus removal system according to embodiments of the present technology.

FIGS. 3A-3H illustrate an elevation view of various configurations of irrigation ports and fluid streams of a thrombus removal system according to embodiments of the present technology.

FIGS. 4A-4D illustrate various embodiments of a thrombus removal system including a saline source, an aspiration system, and one or more controls for controlling irrigation and/or aspiration of the system.

FIGS. 5A-5B illustrate one embodiment of a distal of an elongate medical device.

FIGS. 6A-6E illustrate another embodiment of a distal of an elongate medical device.

FIGS. 7A-7E illustrate another embodiment of a distal of an elongate medical device.

FIGS. 8A-8C illustrate one embodiment of a distal of an elongate medical device.

FIGS. 9A-9B illustrate another embodiment of a distal of an elongate medical device.

FIGS. 10A-10D illustrate one embodiment of a distal end of an elongate medical device with mechanical engagement features configured to function as a cutter.

FIGS. 11A-11D illustrate variations of mechanical engagement features that are disposed to be radially inward-facing, and actuated via a hinge or pivot region to move in a proximal direction.

FIGS. 12A-12B is a variation on a mechanical engagement feature that includes a wire structure that is adapted to act as a lasso.

FIGS. 13A-13D illustrate additional embodiments of mechanical engagement features.

FIGS. 14A-14B illustrate one embodiment of a nested frame approach for a funnel and mechanical engagement features of a thrombus removal device.

FIGS. 15A-15F illustrate examples of array articulation with various examples of mechanical engagement features layer arrangements as described herein.

FIGS. 16A-16G illustrate exemplary orientations and arrangements of mechanical engagement features arrays having one or more layers as described herein.

FIGS. 17A and 17B illustrate an example of a thrombus removal device distal end including a funnel, funnel frame structure, mechanical engagement features frame structure, and mechanical engagement features.

FIGS. 18A-18C illustrate an embodiment of a funnel that includes three mechanical engagement features.

FIGS. 18D-18F illustrate similar views to the embodiment of FIGS. 18A-18C except with a six-mechanical engagement feature design.

FIG. 19A is a side-view of a thrombus removal device including a sheath or delivery catheter.

FIG. 19B is a top view of this open configuration, with the mechanical engagement features resting inside optional “pockets” within the compliant material.

FIG. 19C is another side-view of the thrombus removal device. In this example, the sheath has been advanced distally relative to the thrombus removal device, causing the sheath to engage or contact the mechanical engagement features frame structure and push it distally.

FIG. 19D is a top view of this closed or engaged configuration, showing a view of the pockets 30 within the compliant material and the mechanical engagement features being pulled in towards the aspiration lumen of the device.

FIGS. 20A-20C illustrate an alternative embodiment, in which the mechanical engagement features are concealed by the compliant material when in the open configuration.

FIG. 21 is a flowchart describing a method of assessing a volume of clot removed during treatment.

### SUMMARY OF THE DISCLOSURE

A thrombus removal device is provided, comprising: an elongate catheter having an aspiration lumen; an aspiration source coupled to the aspiration lumen; an expandable funnel coupled to the aspiration lumen and the elongate catheter; at least one mechanical engagement feature disposed within the expandable funnel, wherein the at least one mechanical engagement feature is actuatable to move the at least one mechanical engagement feature within the expandable funnel.

In one aspect, the at least one mechanical engagement feature is actuatable to move the at least one mechanical engagement within the expandable funnel towards the aspiration lumen.

In one aspect, the at least one mechanical engagement feature is actuatable to move the at least one mechanical engagement feature generally radially within the expandable funnel across at least a portion of the expandable funnel.

In one aspect, the at least one mechanical engagement feature comprises a cutting portion.

In one aspect, the at least one mechanical engagement feature comprises a blunted tip.

In one aspect, the device further includes a fluid source; a fluid lumen positioned in the elongate catheter and in fluid communication with the fluid source; a jet orifice positioned near or within the expandable funnel and in fluid communication with the fluid lumen, the jet orifice being configured to provide a fluid stream within the aspiration lumen or within the expandable funnel.

In one aspect, the at least one mechanical engagement feature is actuatable to move the at least one mechanical engagement within the expandable funnel towards the fluid stream.

In one aspect, at least one mechanical engagement feature is in fluid communication with the fluid lumen and is further configured to provide a second fluid stream within the aspiration lumen or within the expandable funnel.

In one aspect, the expandable funnel comprises a funnel frame configured to self-expand the funnel to a fully expanded configuration.

In one aspect, the expandable funnel further comprises a compliant material disposed over at least a portion of the funnel frame.

In one aspect, the device further includes an actuatable frame mechanically coupled to the at least one mechanical engagement feature, the actuatable frame having at least a portion positioned proximally from the funnel frame.

In one aspect, the device further includes a sheath configured to slide axially over the elongate catheter, wherein relative movement therebetween to place the sheath into contact with the actuatable frame moves the at least one mechanical engagement feature within the expandable funnel.

In one aspect, placing the sheath into contact with the actuatable frame does not collapse or reduce a diameter of the expandable funnel.

In one aspect, the at least one mechanical engagement feature is coupled to the funnel frame at a hinge.

In one aspect, a distal portion of the at least one mechanical engagement feature extends from the hinge into the expandable funnel and a proximal portion of the at least one mechanical engagement feature extends outside of the funnel.

In one aspect, the device further includes a sheath configured to slide axially over the elongate catheter, wherein relative movement therebetween to place the sheath into contact with the proximal portion of the at least one mechanical engagement feature moves the distal portion of the at least one mechanical engagement feature about the hinge within the expandable funnel.

In one aspect, the device further includes a sheath configured to rotate around the elongate catheter, wherein rotating the sheath into contact with the proximal portion of the at least one mechanical engagement feature moves the distal portion of the at least one mechanical engagement feature about the hinge within the expandable funnel.

In one aspect, the at least one mechanical engagement feature includes a pair of mechanical engagement features configured to collide at a pinch point when actuated.

In one aspect, the at least one mechanical engagement feature includes a pair of mechanical engagement features configured to create a shearing action when actuated.

In one aspect, the at least one mechanical engagement feature comprises a plurality of mechanical engagement features that are collectively actuated as a group.

In one aspect, the at least one mechanical engagement feature comprises a plurality of mechanical engagement features that are individually and independently actuated.

In one aspect, the at least one mechanical engagement feature comprises a first group of mechanical engagement features that can be actuated independently from a second group of mechanical engagement features.

In one aspect, the at least one mechanical engagement feature comprises a first mechanical engagement feature disposed within the expandable funnel at a first axial position and a second mechanical engagement feature disposed within the expandable funnel at a second axial position distal to the first axial position.

In one aspect, the at least one mechanical engagement feature comprises a plurality of mechanical engagement features configured to move towards a central point within the expandable funnel.

In one aspect, the at least one mechanical engagement feature includes an at-rest configuration in which the at least one mechanical engagement feature is positioned adjacent to or abutting against the compliant material.

In one aspect, the compliant material further comprises at least one pocket corresponding to each of the at least one mechanical engagement features, wherein the at least one mechanical engagement feature includes an at-rest configuration in which the at least one mechanical engagement feature is positioned within its corresponding pocket within the compliant material.

In one aspect, the compliant material further comprises at least one slit corresponding to each of the at least one mechanical engagement features, wherein the at least one mechanical engagement feature includes an at-rest configuration in which the at least one mechanical engagement feature covered by the compliant material and an actuated configuration in which the at least one mechanical engagement feature passes through its corresponding slit to move into the expandable funnel.

In one aspect, actuation of the at least one mechanical engagement feature causes the at least one mechanical engagement feature to pivot within the expandable funnel.

In one aspect, the elongate catheter extends along a longitudinal axis, and further wherein the expandable funnel and at least a portion of the at least one mechanical engagement feature are configured to maintain respective axial positions with respect to the longitudinal axis during actuation of the at least one mechanical engagement feature.

In one aspect, the at least one mechanical feature does not extend beyond a distal end of the expandable funnel.

A medical device is provided, comprising: an elongate catheter; an expandable member positioned at a distal end of the elongate catheter; at least one mechanical engagement feature disposed within the expandable member, wherein the at least one mechanical engagement feature is actuatable to move the at least one mechanical engagement feature within the expandable funnel.

In one aspect, the at least one mechanical engagement feature is actuatable to move the at least one mechanical engagement within the expandable member.

In one aspect, the at least one mechanical engagement feature is actuatable to move the at least one mechanical engagement feature generally radially within the expandable member across at least a portion of the expandable member.

In one aspect, the at least one mechanical engagement feature comprises a cutting portion.

In one aspect, the at least one mechanical engagement feature comprises a blunted tip.

In one aspect, the device further includes a fluid source; a fluid lumen positioned in the elongate catheter and in fluid communication with the fluid source; a jet orifice positioned near or within the expandable funnel and in fluid communication with the fluid lumen, the jet orifice being configured to provide a fluid stream within the expandable member.

In one aspect, the at least one mechanical engagement feature is actuatable to move the at least one mechanical engagement within the expandable funnel towards the fluid stream.

In one aspect, at least one mechanical engagement feature is in fluid communication with the fluid lumen and is further configured to provide a second fluid stream within the aspiration lumen or within the expandable funnel.

In one aspect, the expandable member comprises a frame configured to self-expand the expandable member to a fully expanded configuration.

In one aspect, the expandable member further comprises a compliant material disposed over at least a portion of the frame.

In one aspect, the device further includes an actuatable frame mechanically coupled to the at least one mechanical engagement feature, the actuatable frame having at least a portion positioned proximally from the frame.

In one aspect, the device further includes a sheath configured to slide axially over the elongate catheter, wherein relative movement therebetween to place the sheath into contact with the actuatable frame moves the at least one mechanical engagement feature within the expandable member.

In one aspect, placing the sheath into contact with the actuatable frame does not collapse or reduce a diameter of the expandable member.

In one aspect, the at least one mechanical engagement feature is coupled to the frame at a hinge.

In one aspect, a distal portion of the at least one mechanical engagement feature extends from the hinge into the expandable member and a proximal portion of the at least one mechanical engagement feature extends outside of the expandable member.

In one aspect, the device further includes a sheath configured to slide axially over the elongate catheter, wherein relative movement therebetween to place the sheath into contact with the proximal portion of the at least one mechanical engagement feature moves the distal portion of the at least one mechanical engagement feature about the hinge within the expandable member.

In one aspect, the device further includes a sheath configured to rotate around the elongate catheter, wherein rotating the sheath into contact with the proximal portion of the at least one mechanical engagement feature moves the distal portion of the at least one mechanical engagement feature about the hinge within the expandable member.

In one aspect, the at least one mechanical engagement feature includes a pair of mechanical engagement features configured to collide at a pinch point when actuated.

In one aspect, the at least one mechanical engagement feature includes a pair of mechanical engagement features configured to create a shearing action when actuated.

In one aspect, the at least one mechanical engagement feature comprises a plurality of mechanical engagement features that are collectively actuated as a group.

In one aspect, the at least one mechanical engagement feature comprises a plurality of mechanical engagement features that are individually and independently actuated.

In one aspect, the at least one mechanical engagement feature comprises a first group of mechanical engagement features that can be actuated independently from a second group of mechanical engagement features.

In one aspect, the at least one mechanical engagement feature comprises a first mechanical engagement feature disposed within the expandable member at a first axial position and a second mechanical engagement feature disposed within the expandable member at a second axial position distal to the first axial position.

In one aspect, the at least one mechanical engagement feature comprises a plurality of mechanical engagement features configured to move towards a central point within the expandable member.

In one aspect, the at least one mechanical engagement feature includes an at-rest configuration in which the at least one mechanical engagement feature is positioned adjacent to or abutting against the compliant material.

In one aspect, the compliant material further comprises at least one pocket corresponding to each of the at least one mechanical engagement features, wherein the at least one mechanical engagement feature includes an at-rest configuration in which the at least one mechanical engagement feature is positioned within its corresponding pocket within the compliant material.

In one aspect, the compliant material further comprises at least one slit corresponding to each of the at least one mechanical engagement features, wherein the at least one mechanical engagement feature includes an at-rest configuration in which the at least one mechanical engagement feature covered by the compliant material and an actuated configuration in which the at least one mechanical engagement feature passes through its corresponding slit to move into the expandable member.

In one aspect, actuation of the at least one mechanical engagement feature causes the at least one mechanical engagement feature to pivot within the expandable member.

In one aspect, the elongate catheter extends along a longitudinal axis, and further wherein the expandable funnel and at least a portion of the at least one mechanical engagement feature are configured to maintain respective axial positions with respect to the longitudinal axis during actuation of the at least one mechanical engagement feature.

In one aspect, the at least one mechanical feature does not extend beyond a distal end of the expandable member.

A thrombus removal device is provided, comprising: an elongate catheter having an aspiration lumen and a fluid lumen; an aspiration source coupled to the aspiration lumen; a fluid source coupled to the fluid lumen; an expandable funnel coupled to the aspiration lumen and the elongate catheter; a jet orifice positioned near or within the expandable funnel and in fluid communication with the fluid lumen, the jet orifice being configured to provide a fluid stream within the aspiration lumen or within the expandable funnel; at least one mechanical engagement feature disposed within the expandable funnel, wherein the at least one mechanical engagement feature is actuatable to move the at least one mechanical engagement feature within the expandable funnel towards a plane of the fluid stream.

In one aspect, the at least one mechanical engagement feature is actuatable to move the at least one mechanical engagement within the expandable funnel towards the aspiration lumen.

In one aspect, the at least one mechanical engagement feature is actuatable to move the at least one mechanical engagement feature generally radially within the expandable funnel across at least a portion of the expandable funnel.

In one aspect, the at least one mechanical engagement feature comprises a cutting portion.

In one aspect, the at least one mechanical engagement feature comprises a blunted tip.

In one aspect, the at least one mechanical engagement feature is actuatable to move the at least one mechanical engagement within the expandable funnel towards the fluid stream.

In one aspect, at least one mechanical engagement feature is in fluid communication with the fluid lumen and is further configured to provide a second fluid stream within the aspiration lumen or within the expandable funnel.

In one aspect, the expandable funnel comprises a funnel frame configured to self-expand the funnel to a fully expanded configuration.

In one aspect, the expandable funnel further comprises a compliant material disposed over at least a portion of the funnel frame.

In one aspect, the device further includes an actuatable frame mechanically coupled to the at least one mechanical engagement feature, the actuatable frame having at least a portion positioned proximally from the funnel frame.

In one aspect, the device further includes a sheath configured to slide axially over the elongate catheter, wherein relative movement therebetween to place the sheath into contact with the actuatable frame moves the at least one mechanical engagement feature within the expandable funnel.

In one aspect, placing the sheath into contact with the actuatable frame does not collapse or reduce a diameter of the expandable funnel.

In one aspect, the at least one mechanical engagement feature is coupled to the funnel frame at a hinge.

In one aspect, a distal portion of the at least one mechanical engagement feature extends from the hinge into the expandable funnel and a proximal portion of the at least one mechanical engagement feature extends outside of the funnel.

In one aspect, a sheath configured to slide axially over the elongate catheter, wherein relative movement therebetween to place the sheath into contact with the proximal portion of the at least one mechanical engagement feature moves the distal portion of the at least one mechanical engagement feature about the hinge within the expandable funnel.

In one aspect, the device further includes a sheath configured to rotate around the elongate catheter, wherein rotating the sheath into contact with the proximal portion of the at least one mechanical engagement feature moves the distal portion of the at least one mechanical engagement feature about the hinge within the expandable funnel.

In one aspect, the at least one mechanical engagement feature includes a pair of mechanical engagement features configured to collide at a pinch point when actuated.

In one aspect, the at least one mechanical engagement feature includes a pair of mechanical engagement features configured to create a shearing action when actuated.

In one aspect, the at least one mechanical engagement feature comprises a plurality of mechanical engagement features that are collectively actuated as a group.

In one aspect, the at least one mechanical engagement feature comprises a plurality of mechanical engagement features that are individually and independently actuated.

In one aspect, the at least one mechanical engagement feature comprises a first group of mechanical engagement features that can be actuated independently from a second group of mechanical engagement features.

In one aspect, the at least one mechanical engagement feature comprises a first mechanical engagement feature disposed within the expandable funnel at a first axial position and a second mechanical engagement feature disposed within the expandable funnel at a second axial position distal to the first axial position.

In one aspect, the at least one mechanical engagement feature comprises a plurality of mechanical engagement features configured to move towards a central point within the expandable funnel.

In one aspect, the at least one mechanical engagement feature includes an at-rest configuration in which the at least one mechanical engagement feature is positioned adjacent to or abutting against the compliant material.

In one aspect, the compliant material further comprises at least one pocket corresponding to each of the at least one mechanical engagement features, wherein the at least one mechanical engagement feature includes an at-rest configuration in which the at least one mechanical engagement feature is positioned within its corresponding pocket within the compliant material.

In one aspect, the compliant material further comprises at least one slit corresponding to each of the at least one mechanical engagement features, wherein the at least one mechanical engagement feature includes an at-rest configuration in which the at least one mechanical engagement feature covered by the compliant material and an actuated configuration in which the at least one mechanical engagement feature passes through its corresponding slit to move into the expandable funnel.

In one aspect, actuation of the at least one mechanical engagement feature causes the at least one mechanical engagement feature to pivot within the expandable funnel.

In one aspect, the elongate catheter extends along a longitudinal axis, and further wherein the expandable funnel and at least a portion of the at least one mechanical engagement feature are configured to maintain respective axial positions with respect to the longitudinal axis during actuation of the at least one mechanical engagement feature.

In one aspect, the at least one mechanical feature does not extend beyond a distal end of the expandable funnel.

A thrombus removal device, comprising: an elongate catheter having an aspiration lumen; an aspiration source coupled to the aspiration lumen; an expandable funnel coupled to the aspiration lumen and the elongate catheter, the expandable funnel comprising a frame; at least one mechanical engagement feature disposed within the expandable funnel; and a sheath slidably disposed along an outside of the elongate catheter, wherein relative movement between the sheath and the elongate catheter places the sheath into contact with a portion of the expandable funnel to cause the at least one mechanical engagement feature to move within the funnel.

In one aspect, the at least one mechanical engagement feature is actuatable to move the at least one mechanical engagement within the expandable funnel towards the aspiration lumen.

In one aspect, the at least one mechanical engagement feature is actuatable to move the at least one mechanical engagement feature generally radially within the expandable funnel across at least a portion of the expandable funnel.

In one aspect, the at least one mechanical engagement feature comprises a cutting portion.

In one aspect, the at least one mechanical engagement feature comprises a blunted tip.

In one aspect, the device further includes a fluid source; a fluid lumen positioned in the elongate catheter and in fluid communication with the fluid source; a jet orifice positioned near or within the expandable funnel and in fluid communication with the fluid lumen, the jet orifice being configured to provide a fluid stream within the aspiration lumen or within the expandable funnel.

In one aspect, the at least one mechanical engagement feature is actuatable to move the at least one mechanical engagement within the expandable funnel towards the fluid stream.

In one aspect, at least one mechanical engagement feature is in fluid communication with the fluid lumen and is further configured to provide a second fluid stream within the aspiration lumen or within the expandable funnel.

In one aspect, the expandable funnel comprises a funnel frame configured to self-expand the funnel to a fully expanded configuration.

In one aspect, the expandable funnel further comprises a compliant material disposed over at least a portion of the funnel frame.

In one aspect, the device further includes an actuatable frame mechanically coupled to the at least one mechanical engagement feature, the actuatable frame having at least a portion positioned proximally from the funnel frame.

In one aspect, the at least one mechanical engagement feature is coupled to the funnel frame at a hinge.

In one aspect, a distal portion of the at least one mechanical engagement feature extends from the hinge into the expandable funnel and a proximal portion of the at least one mechanical engagement feature extends outside of the funnel.

In one aspect, the at least one mechanical engagement feature includes a pair of mechanical engagement features configured to collide at a pinch point when actuated.

In one aspect, the at least one mechanical engagement feature includes a pair of mechanical engagement features configured to create a shearing action when actuated.

In one aspect, the at least one mechanical engagement feature comprises a plurality of mechanical engagement features that are collectively actuated as a group.

In one aspect, the at least one mechanical engagement feature comprises a plurality of mechanical engagement features that are individually and independently actuated.

In one aspect, the at least one mechanical engagement feature comprises a first group of mechanical engagement features that can be actuated independently from a second group of mechanical engagement features.

In one aspect, the at least one mechanical engagement feature comprises a first mechanical engagement feature disposed within the expandable funnel at a first axial position and a second mechanical engagement feature disposed within the expandable funnel at a second axial position distal to the first axial position.

In one aspect, the at least one mechanical engagement feature comprises a plurality of mechanical engagement features configured to move towards a central point within the expandable funnel.

In one aspect, the at least one mechanical engagement feature includes an at-rest configuration in which the at least one mechanical engagement feature is positioned adjacent to or abutting against the compliant material.

In one aspect, the compliant material further comprises at least one pocket corresponding to each of the at least one mechanical engagement features, wherein the at least one mechanical engagement feature includes an at-rest configuration in which the at least one mechanical engagement feature is positioned within its corresponding pocket within the compliant material.

In one aspect, the compliant material further comprises at least one slit corresponding to each of the at least one mechanical engagement features, wherein the at least one mechanical engagement feature includes an at-rest configuration in which the at least one mechanical engagement feature covered by the compliant material and an actuated configuration in which the at least one mechanical engagement feature passes through its corresponding slit to move into the expandable funnel.

In one aspect, actuation of the at least one mechanical engagement feature causes the at least one mechanical engagement feature to pivot within the expandable funnel.

In one aspect, the elongate catheter extends along a longitudinal axis, and further wherein the expandable funnel and at least a portion of the at least one mechanical engagement feature are configured to maintain respective axial positions with respect to the longitudinal axis during actuation of the at least one mechanical engagement feature.

In one aspect, the at least one mechanical feature does not extend beyond a distal end of the expandable funnel.

A method of removing thrombus from a patient, comprising: inserting a thrombectomy catheter into the patient; expanding a distal expandable member of the catheter adjacent to a target thrombus; aspirating the target thrombus into the distal expandable member; actuating at least one mechanical engagement feature within the funnel to contact the target thrombus; and aspirating the target thrombus out of the thrombectomy catheter.

In one aspect, the method further includes directing at least two intersecting jet streams into the target thrombus within the distal expandable member.

In one aspect, actuating the at least one mechanical engagement feature further comprises cutting the target thrombus with the at least one mechanical engagement feature.

In one aspect, actuating the at least one mechanical engagement feature further comprises pinching the target thrombus with the at least one mechanical engagement feature.

In one aspect, actuating the at least one mechanical engagement feature further comprises shearing the target thrombus with the at least one mechanical engagement feature.

In one aspect, actuating the at least one mechanical engagement feature further comprises moving the at least one engagement feature towards an aspiration lumen of the thrombectomy catheter.

In one aspect, actuating the at least one mechanical engagement feature further comprises moving the at least one engagement feature radially across the distal expandable member.

In one aspect, actuating the at least one mechanical engagement feature further comprises moving the at least one engagement feature towards a plane of the intersecting jet streams.

A thrombus removal device, comprising: an elongate shaft having a distal end; at least one aspiration lumen in the elongate shaft; an expandable funnel disposed at or near the distal end, the funnel comprising: a nested frame structure comprising an actuatable frame structure proximal to a funnel frame structure; at least one mechanical engagement feature coupled to or integral with the actuatable frame structure; and a sheath slideably disposed over the elongate shaft, wherein the sheath is configured to engage the actuatable frame structure to actuate the at least one mechanical engagement feature between an open or expanded configuration and a closed or actuated configuration.

In one aspect, engaging the actuatable frame structure with the sheath does not cause engagement between the sheath and the funnel frame structure.

In one aspect, the device further includes a compliant material disposed around the funnel frame structure and at least a portion of the actuatable frame structure.

In one aspect, the compliant material is not disposed around the at least one mechanical engagement feature.

In one aspect, the device further includes at least one pocket disposed in the compliant material, the pocket being configured to receive the at least one mechanical engagement feature when the at least one mechanical engagement feature is in the open configuration.

In one aspect, the device further includes at least one slit in the compliant material over each of the at least one pockets, the at least one slit being configured to allow the mechanical engagement feature to emerge from the at least one pocket when being actuated between the open configuration and closed or actuated configuration.

In one aspect, the device further includes a cutting or serrated edge on the at least one mechanical engagement feature.

In one aspect, at least one mechanical engagement feature is directed towards the aspiration lumen in the closed or actuated configuration.

A method for removing a thrombus from a blood vessel of a patient with a thrombus removal device is provided, the method comprising: introducing a distal portion of a thrombus removal device to a thrombus location in a blood vessel; retracting a sheath along an elongate shaft of the thrombus removal device to expand a funnel at the thrombus location; operating an aspiration source of the thrombus removal device to at least partially capture a thrombus in the funnel; advancing the sheath along the elongate shaft to engage a frame structure of the funnel and to cause at least one mechanical engagement feature of the funnel to actuate from an open configuration to a closed configuration.

In one aspect, actuating the at least one mechanical engagement feature from the open configuration to the closed configuration does not collapse the funnel.

In one aspect, the at least one mechanical engagement feature moves towards an aspiration lumen of the elongate shaft in the closed configuration.

A thrombus removal device is provided, comprising: an elongate shaft; at least one aspiration lumen in the elongate shaft; a funnel disposed at a distal end of the elongate shaft; an array of mechanical engagement features operably positioned within the funnel, the array comprising one or more axially spaced mechanical engagement feature layers actuatable to engage thrombus material.

A method for removing thrombus from a blood vessel of a patient with a thrombus removal device is provided, the method comprising: obtaining a pre-treatment image representative of a thrombus; introducing a distal portion of an elongate catheter in a blood vessel to a target location near the thrombus; operating an aspiration source of the elongate catheter; removing the thrombus from the patient with the aspiration source through the thrombus removal device; and determining a volume of the thrombus removed from the patient.

In one aspect, the method further comprises calculating a pre-treatment volume of the thrombus from the pre-treatment image.

In one aspect, determining the volume further comprises: obtaining a post-treatment image of the thrombus; calculating a post-treatment volume of the thrombus from the post-treatment image; and comparing the post-treatment volume to the pre-treatment volume.

In one aspect, the method further comprises measuring a parameter related to removing the thrombus.

In one aspect, measuring the parameter further comprises measuring a flow rate or pressure.

In one aspect, determining the volume further comprises estimating or calculating the volume of thrombus removed based on the measured parameter.

In one aspect, the method further comprises calculating a pre-treatment volume of the thrombus from the pre-treatment image; comparing the estimated or calculated volume of thrombus removed to the pre-treatment volume.

In one aspect, the method further comprises moving the distal portion of the catheter to another location near another thrombus; and removing the another thrombus.

In one aspect, the method further comprises generating an indicator that sufficient thrombus has been removed; and displaying a representation of the indicator.

In one aspect, the indicator is based on one of the thrombus, the another thrombus, and a combination thereof.

A console for controlling a thrombectomy catheter is provided, comprising: a pump for controlling aspiration through the thrombectomy catheter; a sensor for measuring a volume of thrombus removed through the thrombectomy catheter; and a processor including instructions for determining a pre-treatment volume of thrombus in a treatment location and comparing the volume of thrombus removed to the pre-volume.

A thrombus removal device console is provided, comprising: an aspiration source; a cannister fluidly coupled to the aspiration source, the cannister being configured to be fluidly coupled to an aspiration lumen of a thrombus removal device; a sensor disposed in the cannister, the sensor being configured to characterize or determine an amount of fluid or biological materials removed from a patient.

In one aspect, the sensor comprises a weight scale.

In one aspect, the sensor comprises a camera.

In one aspect, the canister further comprises a filter configure to allow blood and/or fluid to drain from the cannister but not clots.

In one aspect, the console further includes a fluid source and a second sensor disposed on or in the fluid source.

In one aspect, the second sensor comprises a flow sensor or a weight scale configured to measure a volume or weight of fluid delivered from the fluid source.

### DETAILED DESCRIPTION

This application is related to disclosure in International Application No. PCT/US2021/020915, filed Mar. 4, 2021 (the '915 application), and International Application No. PCT/US2022/033024, filed Jun. 10, 2022 (the '024 application), the disclosures of which are incorporated by reference herein for all purposes. The '915 and '024 applications describes general mechanisms for capturing and removing a clot. By example, multiple fluid streams are directed toward the clot to fragment the material.

The present technology is generally directed to thrombus removal systems and associated methods. A system configured in accordance with an embodiment of the present technology can include, for example, an elongated catheter having a distal portion configured to be positioned within a blood vessel of the patient, a proximal portion configured to be external to the patient, a fluid delivery mechanism configured to fragment the thrombus with pressurized fluid, an aspiration mechanism configured to aspirate the fragments of the thrombus, and one or more lumens extending at least partially from the proximal portion to the distal portion.

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the examples but are not described in detail with respect to the figures.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

Reference throughout this specification to relative terms such as, for example, “generally,” “approximately,” and “about” are used herein to mean the stated value plus or minus 10%.

Although some embodiments herein are described in terms of thrombus removal, it will be appreciated that the present technology can be used and/or modified to remove other types of emboli that may occlude a blood vessel, such as fat, tissue, or a foreign substance. Additionally, although some embodiments herein are described in the context of thrombus removal from a pulmonary artery (e.g., pulmonary embolectomy), the technology may be applied to removal of thrombi and/or emboli from other portions of the vasculature (e.g., in neurovascular, coronary, within chambers of the heart, or peripheral applications). Moreover, although some embodiments are discussed in terms of maceration of a thrombus with a fluid, the present technology can be adapted for use with other techniques for breaking up a thrombus into smaller fragments or particles (e.g., ultrasonic, mechanical, enzymatic, etc.).

The headings provided herein are for convenience only and do not interpret the scope or meaning of the claimed present technology.

### Systems for Thrombus Removal

As provided above, the present technology is generally directed to thrombus removal systems. Such systems include an elongated catheter having a distal portion positionable within a blood vessel of the patient (e.g., an artery or vein), a proximal portion positionable outside the patient's body, a fluid delivery mechanism configured to fragment the thrombus with pressurized fluid, an aspiration mechanism configured to aspirate the fragments of the thrombus, and one or more lumens extending at least partially from the proximal portion to the distal portion. In some embodiments, the systems herein are configured to engage a thrombus in a patient's blood vessel, break the thrombus into small fragments, and aspirate the fragments out of the patient's body. The pressurized fluid streams (e.g., jets) function to cut or macerate thrombus, before, during, and/or after at least a portion of the thrombus has entered the aspiration lumen or a funnel of the system. Fragmentation helps to prevent clogging of the aspiration lumen and allows the thrombus removal system to macerate large, firm clots that otherwise could not be aspirated. As used herein, “thrombus” and “embolism” are used somewhat interchangeably in various respects. Typically a thrombus is a portion of clotted blood that has stopped moving through the vasculature and is lodged or stuck and the emboli is a portion of clotted blood that is moving in the vasculature that can eventually become a thrombus and additionally seed a larger thrombus either by collecting other emboli or blood clotting on the thrombus.

It should be appreciated that while the description may refer to removal of “thrombus,” this should be understood to encompass removal of thrombus fragments and other emboli as provided herein.

According to embodiments of the present technology, a fluid delivery mechanism can provide a plurality of fluid streams (e.g., jets) to fluid apertures of the thrombus removal system for macerating, cutting, fragmenting, pulverizing and/or urging thrombus to be removed from a proximal portion of the thrombus removal system. The thrombus removal system can include an aspiration lumen extending at least partially from the proximal portion to the distal portion of the thrombus removal system that is adapted for fluid communication with an aspiration pump (e.g., vacuum source). In operation, the aspiration pump may generate a volume of lower pressure within the aspiration lumen near the proximal portion of the thrombus removal system, urging aspiration of thrombus from the distal portion to the proximal portion.

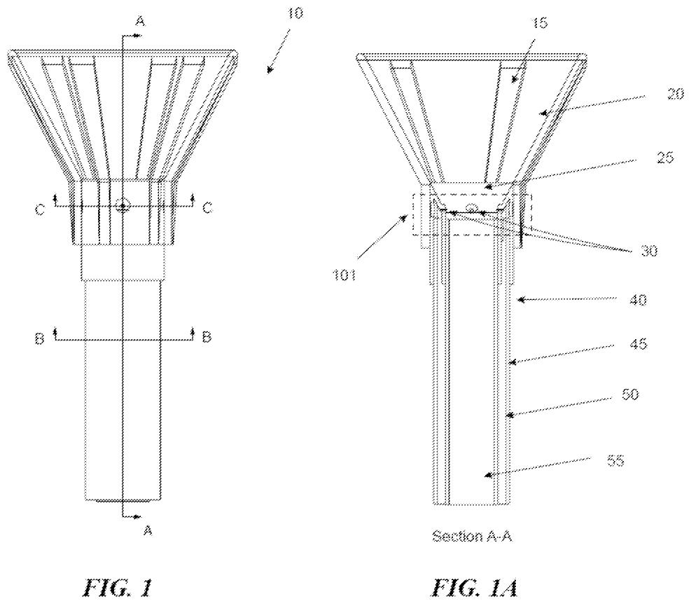

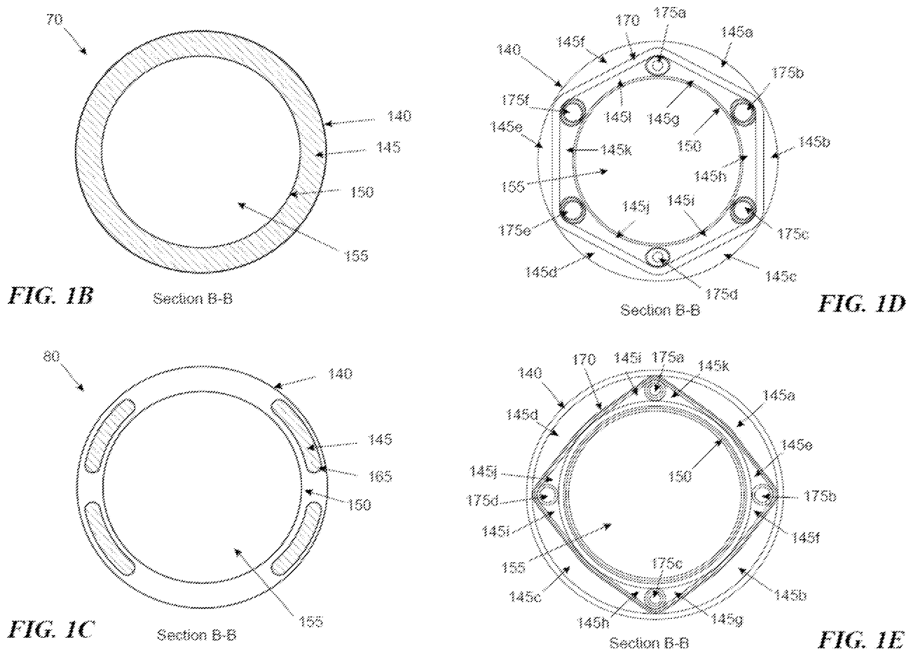

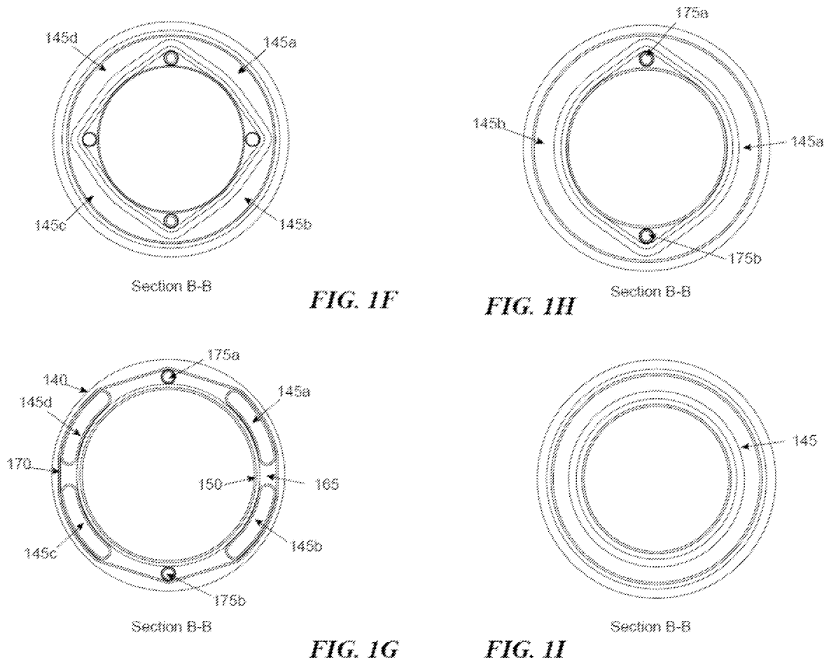

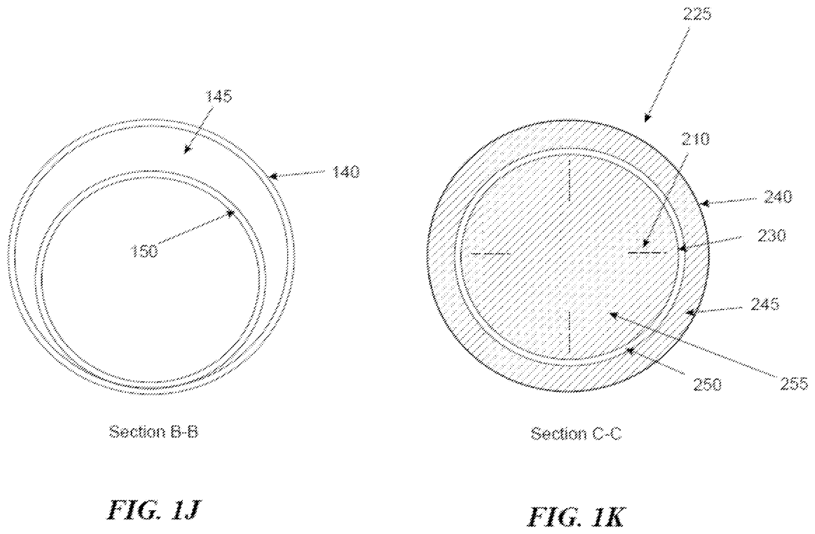

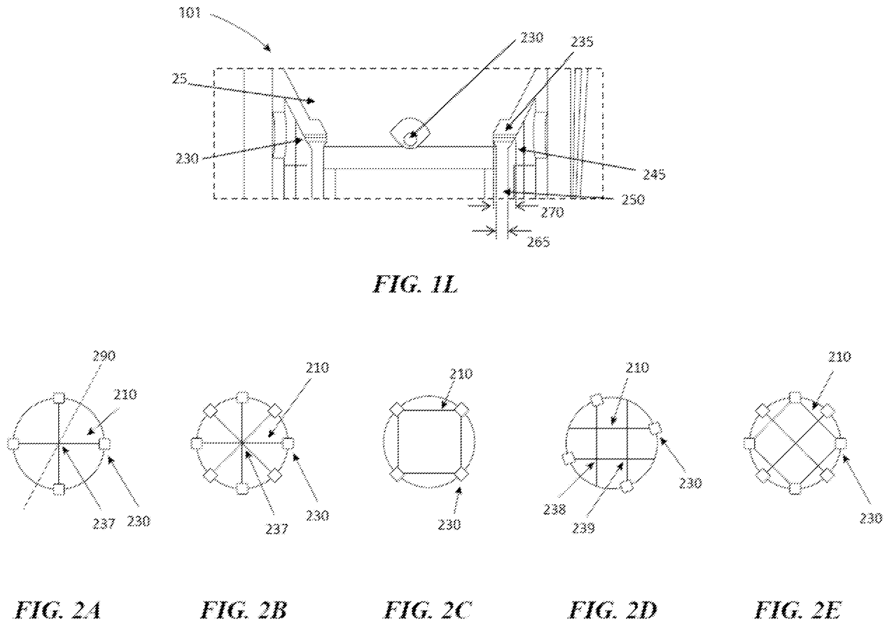

FIG. 1 illustrates a distal portion 10 of a thrombus removal system according to an embodiment of the present technology. FIG. 1A Section A-A illustrates an elevation sectional view of the distal portion. The example section A-A in FIG. 1A depicts a funnel 20 that is positioned at the distal end of the distal portion 10, the funnel adapted to engage with thrombus and/or a tissue (e.g., vessel) wall to aid in thrombus collection, fragmentation, and/or removal. The funnel can have a variety of shapes and constructions as would be understood by one of skill from the description herein. The example section A-A in FIG. 1A depicts a double walled thrombus removal device construction having an outer wall/tube 40 and an inner wall/tube 50. An aspiration lumen 55 is formed by the inner wall 50 and is centrally located. A generally annular volume forms at least one fluid lumen 45 between the outer wall 40 and the inner wall 50. The fluid lumen 45 is adapted for fluid communication with the fluid delivery mechanism. One or more apertures (e.g., nozzles, orifices, or ports) 30 are positioned in the thrombus removal system to be in fluid communication with the fluid lumen 45 and an irrigation manifold 25. In operation, the ports 30 are adapted to direct (e.g., pressurized) fluid toward thrombus that is engaged with the distal portion 10 of the thrombus removal system.

In various embodiments, the system can have an average flow velocity within the fluid lumen of up to 20 m/s to achieve consistent and successful aspiration of clots. In some embodiments, the fluid source itself can be delivered in a pulsed sequence or a preprogrammed sequence that includes some combination of pulsatile flow and constant flow to deliver fluid to the jets. In these embodiments, while the average pulsed fluid velocity may be up to 20 m/s, the peak fluid velocity in the lumen may be up to 30 m/s or more during the pulsing of the fluid source. In some embodiments, the jets or apertures have an aperture size ranging between 0.005″ to 0.020″ to avoid undesirable spraying of fluid. In some embodiments, the system can have a minimum vacuum or aspiration pressure of 15 inHg, to remove target clots after they have been macerated or broken up with the jets described above.

The thrombus removal system can be sized and configured to access and remove thrombi in various locations or vessels within a patient's body. It should be understood that while the dimensions of the system may vary depending on the target location, generally similar features and components described herein may be implemented in the thrombus removal system regardless of the application. For example, a thrombus removal system configured to remove pulmonary embolism (PE) from a patient may have an outer wall/tube with a size of approximately 11-13 Fr, or preferably 12 Fr, and an inner wall/tube with a size of 7-9 Fr, or preferably 8 Fr. A deep vein thrombosis (DVT) device, on the other hand, may have an outer wall/tube with a size of approximately 9-11 Fr, or preferably 10 Fr, and an inner wall/tube with a size of 6-9 Fr, or preferably 7.5 Fr. Applications are further provided for ischemic stroke and peripheral embolism applications.

Section B-B of FIG. 1B illustrates in plan view a portion of the thrombus removal system that is proximal to the funnel and irrigation manifold. Section B-B depicts an outer wall 140, an inner wall 150, an aspiration lumen 155 and a fluid lumen 145. In some embodiments, in cross-section the aspiration lumen 155 is generally circular and the fluid lumen 145 is generally annular in shape (e.g., cross-section 70). It will be appreciated that alternative constructions and/or arrangements of the inner wall 150 and the outer wall 140 produce variations in cross-sectional shape of the aspiration and fluid lumens 155 and 145. For example, the inner wall 150 can be shaped to form an aspiration lumen 155 that, in cross-section, is generally oval, circular, rectilinear, square, pentagonal, or hexagonal. The inner and outer walls 150 and 140 can be shaped and arranged to form a fluid lumen 145 that, in cross-section, is generally crescent-shaped, diamond shaped, or irregularly shaped. For example, referring to FIG. 1C Section B-B, the region between the inner wall 150 and the outer wall 140 can include one or more wall structures 165 that form respective fluid lumens 145 (e.g., as in cross-section 80). The wall structures 165 can be formed by lamination between the outer and inner walls 140 and 150, or by a multi-lumen extrusion that forms a plurality of the wall structures.

Section B-B of FIGS. 1D-1H illustrate additional examples of a portion of the thrombus removal system that is proximal to the funnel and irrigation manifold. Similar to the embodiments described above, the portion in these examples can include an outer wall 140, an inner wall 150, and an aspiration lumen 155. Additionally, the illustrated portion of the thrombus removal system can include a middle wall 170 disposed between the outer wall 140 and the inner wall 150. The middle wall 170 enables further segmentation of the annular space between the inner wall and outer wall into a plurality of distinct fluid lumens and/or auxiliary lumens. For example, referring to FIG. 1D, the middle wall can be generally hexagon shaped, and the annular space can include a plurality of fluid lumens 145a-141 and a plurality of auxiliary lumens 175a-175f. As shown in FIG. 1D, the fluid lumens can be formed by some combination of the outer wall 140 and the middle wall 170, or between the middle wall 170, the inner wall 150, and two of the auxiliary lumens. For example, fluid lumen 145a is formed in the space between outer wall 140 and middle wall 170. However, fluid lumen 145g is formed in the space between middle wall 170, inner wall 150, auxiliary lumen 175a, and auxiliary lumen 175b. Generally, the fluid lumens are configured to carry a flow of fluid such as saline from a saline source of the system to one or more ports/apertures/orifices of the system. The auxiliary lumens can be configured for a number of functions. In some embodiments, the auxiliary lumens can be coupled to the fluid/saline source and to the apertures to be used as additional fluid lumens. In other embodiments, the auxiliary lumens can be configured as steering ports and can include a guide wire or steering wire within the lumen for steering of the thrombus removal system. Additionally, in other embodiments, the auxiliary lumens can be configured to carry electrical, mechanical, or fluid connections to one or more sensors. For example, the system may include one or more electrical, optical, or fluid based sensors disposed along any length of the system. The sensors can be used during therapy to provide feedback for the system (e.g., sensors can be used to detect clogs to initiate a clog removal protocol, or to determine the proper therapy mode based on sensor feedback such as jet pulse sequences, aspiration sequences, and or proper functioning of the system, etc.). The auxiliary ports can therefore be used to connect to the sensors, e.g., by electrical connection, optical connection, mechanical/wire connection, and/or fluid connection. It is also contemplated that the fluid and auxiliary lumens can be configured to carry and deliver other fluids, such as thrombolytics or radio-opaque contrast injections to the target tissue site during treatment.

It should be understood that in some embodiments, all the fluid lumens are fluidly connected to all of the jets or apertures of the thrombus removal device. Therefore, when a flow of fluid is delivered from the fluid lumen(s) to the jets, all jets are activated with a jet of fluid at once. However, it should also be understood that in some embodiments, the fluid lumens are separate or distinct, and these distinct fluid lumens may be fluidly coupled to one or more jets but not to all jets of the device. In these embodiments, a subset of the jets can be controlled by delivering fluid only to the fluid lumens that are coupled to that subset of jets. This enables additional functionality in the device, in which specific jets can be activated in a user defined or predetermined order.

In various embodiments, the fluid pressure is generated at the pump (at the console or handle). The fluid is accelerated as it exits through the ports at the distal end and is directed to the target clot. In this way a wider variety of cost-effective components can be used to form the catheter while still maintaining a highly-effective device for clot removal. Additional details are provided below.

Section B-B of FIG. 1E illustrates another embodiment of the portion of the thrombus removal system that is proximal to the funnel and irrigation manifold. Similar to the embodiment of FIG. 1D, this embodiment also includes a middle wall 170. However, the middle wall in this example is generally square shaped, facilitating the formation of fluid lumens 145a-145k and auxiliary lumens 175a-175d. The example illustrated in section B-B of FIG. 1F is similar to that of the embodiment of FIG. 1E, however this embodiment includes only fluid lumens 145a-145d. The fluid lumens 145e-145k from the embodiment of FIG. 1E are not used as fluid lumens in this embodiment. They can be, for example, empty lumens, vacuum, filled with an insulative material, and/or filled with a radio-opaque material or any other material that may help visualize the thrombus removal system during therapy. The embodiment 1F includes the same four auxiliary reports as illustrated and described in the embodiment of FIG. 1E.

Section B-B of FIG. 1G illustrates another example of a portion of the thrombus removal system that is proximal to the funnel and irrigation manifold. Similar to the embodiments described above, the illustrated portion of the thrombus removal system can include a middle wall 170 disposed between the outer wall 140 and the inner wall 150. However, this embodiment includes four distinct fluid lumens 145a-145d formed by wall structures 165. As with the embodiment of FIG. 1C, the wall structures 165 can be formed by lamination between the outer and inner walls 140 and 150, or by a multi-lumen extrusion that forms a plurality of the wall structures. As shown, this embodiment can include a pair of auxiliary lumens 175a and 175b, which can be used, for example, for steering or for sensor connections as described above.

Section B-B of FIG. 1H is another similar embodiment in which the middle wall and outer wall can be used to form fluid lumens 145a and 145b. Auxiliary lumens 175a and 175b can be formed in the space between the middle wall and the inner wall. It should be understood that the middle wall can contact the outer wall to create independent fluid lumens 145a and 145b. However, in other embodiments, it should be understood that the middle wall may not contact the outer wall, which would facilitate a single annular fluid lumen, such as is shown by fluid lumen 145 in Section B-B of FIG. 1I. In another embodiment, as shown in Section B-B of FIG. 1J, the inner wall 150 and the outer wall 140 may not be concentric, which facilitates formation of an annular space and/or fluid lumen 145 that is thicker or wider on one side of the device relative to the other side. As shown in FIG. 1J, a distance between the exemplary outer wall 140 and inner wall at the top (e.g., 12 o'clock) portion of the device is larger than a distance between the outer wall and inner wall at the bottom (e.g., 6 o'clock) portion of the device.

Section C-C of FIG. 1K illustrates in plan view a portion of the thrombus removal system comprising an irrigation manifold 225. Section C-C depicts an outer wall 240, an inner wall 250, a fluid lumen 245, an aspiration lumen 255, and ports 230 for directing respective fluid streams 210.

Detail View 101 of FIG. 1L illustrates a section view in elevation of a portion of the irrigation manifold 25 that includes a plurality of ports 230 that are formed within an inner wall 250. In some embodiments, a thickness of one or more walls of the thrombus removal system may be varied along its axial length and/or its circumference. As shown in Detail View 101, inner wall 250 has a first thickness 265 in a region 250 that is proximal to the irrigation manifold 25, and a second thickness 270 in a region 235 that includes the ports 230. In some embodiments, the second thickness 270 is greater than the first thickness 265. The first thickness 265 can correspond to a general wall thickness of the inner wall 50 and/or of the outer wall 40, which can be from about 0.10 mm to about 0.60 mm, or any value within the aforementioned range. The second thickness 270 can be from about 0.20 mm to about 0.70 mm, from about 0.70 mm to about 0.90 mm, or from about 0.90 mm to about 1.20 mm. The second thickness 270 can be any value within the aforementioned range. The dimension of the second thickness 270 can be selected to provide a fluid path through the ports 230 that produces a generally laminar flow for a fluid stream that is directed therethrough, when the fluid delivery mechanism supplies fluid via the fluid lumen 245 at a typical operating pressure. Such operating pressure can be from about 10 psi to about 60 psi, from about 60 psi to about 100 psi, or from about 100 psi to about 150 psi. The operating pressure of the fluid delivery mechanism can be any value within the aforementioned range of values. In some embodiments, the fluid delivery mechanism is operated in a high pressure mode, having a pressure from about 150 psi to about 250 psi, from about 250 psi to about 350 psi, from about 350 psi to about 425 psi, or from about 425 psi to about 500 psi, or up to 1,000 psi. The operating pressure of the fluid delivery mechanism in the high pressure mode can be any value within the aforementioned range of values.

The manifold is configured to increase a fluid pressure and/or flow rate of the fluid. When fluid is provided by the fluid delivery mechanism to the fluid lumen(s) at a first pressure and/or a first flow rate, the manifold is configured to increase the pressure of the fluid to a second pressure and/or is configured to increase the flow rate of the fluid to a second flow rate. The second pressure and/or second fluid rate can be higher than the first pressure and/or first flow rate. As a result, the manifold can be configured to increase the relatively low operating pressures and/or flow rates generated by the fluid delivery mechanism to the relatively high pressures and/or high flow rates generated by the ports/fluid streams.

In some embodiments, a profile (cross-sectional dimension) of a port 230 varies along its length (e.g., is non-cylindrical). A variation in the cross-sectional dimension of the port may alter and/or adjust a characteristic of fluid flow along the port 230. For example, a reduction in cross-sectional dimension may accelerate a flow of fluid through the port 230 (for a given volume of fluid). In some embodiments, a port 230 may be conical along its length (e.g., tapered), such that its smallest dimension is positioned at the distal end of the port 230, where distal is with respect to a direction of fluid flow.

In some embodiments, the port 230 is formed to direct the fluid flow along a selected path. FIGS. 2A-2E illustrate various embodiments of arrangements of ports 230 for directing respective fluid streams 210. In some embodiments, such as those shown in FIGS. 2A and 2B, at least two ports 230 are arranged to produce (e.g., respective) fluid streams 210 that intersect at an intersection region 237 of the thrombus removal system. An intersection region 237 can be a region of increased fluid momentum, turbulence, shear, and/or energy transfer, which multiply with respect to individual fluid streams that are not directed to combine at the intersection. The increased fluid momentum and/or energy transfer at an intersection may advantageously fragment thrombus more efficiently and/or quickly. As described above, the fluid streams can be configured to accelerate and cause cavitation and/or other effects to further add to breaking up of the target clot. In some embodiments, an intersection region can be formed from at least 2, at least 3, at least 4, at least 5, at least 6, at least 7, at least 8, at least 9, or at least 10 fluid streams 210. An intersection region can be generally near a central axis 290 of the thrombus removal system (e.g., 237), or away from the central axis (e.g., 238 and 239 in the embodiment of FIG. 2D). In some embodiments, at least two intersection regions (e.g., 238 and 239) are formed. In some embodiments, one or more ports 230 are arranged to direct a fluid stream 210 along an oblique angle with respect to the central axis of the thrombus removal system. An operating pressure of the fluid delivery mechanism may be selected to approach a minimum targeted fluid velocity for a fluid stream 210 that is delivered from a port 230. The targeted fluid velocity for a fluid stream 210 can be about 5 meters/second (m/s), about 8 m/s, about 10 m/s, about 12 m/s, or about 15 m/s. Additionally, the targeted fluid velocities in some embodiments can be in the range above 15 m/s to up to 150 m/s. At these higher velocities (e.g. above 15 m/s, or alternatively above 20 m/s), the fluid streams may be configured to generate cavitation in a target thrombus or tissue. It has been found that with fluid exiting from the ports to these flow rates a cavitation effect can be created in the focal area of the intersecting or colliding fluid streams, or additionally at a boundary of one or more of the fluid streams. While the exact specifications may change based on the catheter size, in general, at least one of the fluid streams should be accelerated to such a high velocity to create cavitation as described in detail below. The targeted fluid velocity for fluid stream 210 can be any value within the range of aforementioned values. In some embodiments, at least two ports 230 are adapted to deliver respective fluid streams at different fluid velocities (i.e. speed and direction), for a given pressure of the fluid delivery mechanism. In some embodiments, at least two ports 230 are adapted to deliver respective fluid streams at the substantially the same fluid velocities, for a given pressure of the fluid delivery mechanism. In some embodiments, one port is adapted to deliver fluid at high velocity and the respective one or more other ports is adapted to deliver fluid at relatively lower velocities. Advantageously, an increased cross-sectional area of the fluid lumen 145 reduces a required operating pressure of the fluid delivery mechanism to achieve a targeted fluid velocity of the fluid streams.

In some embodiments, the fluid streams are configured to create angular momentum that is imparted to a thrombus. In some examples, angular momentum is imparted on the thrombus by application of a) at least one fluid stream 210 that is directed at an oblique angle from a port 230, and/or b) at least two fluid streams 210 that have different fluid velocities. For example, fluid streams that cross near each other but do not necessarily intersect may create a “swirl” or rotational energy on the clot material. Advantageously, angular momentum produced in a thrombus may impart a (e.g., centrifugal) force that assists in fragmentation and removal of the thrombus. Rotating of the clot may enhance delivery of the clot material to the jets. By example, with a large, amorphous clot the soft material may be easily aspirated or broken up by the fluid streams whereas tough fibrin may be positioned away from the fluid streams. Rotating or swirling of the clot moves the material around so the harder clot material is presented to the jets. The swirling may also further break up the clot as it is banged inside the funnel.

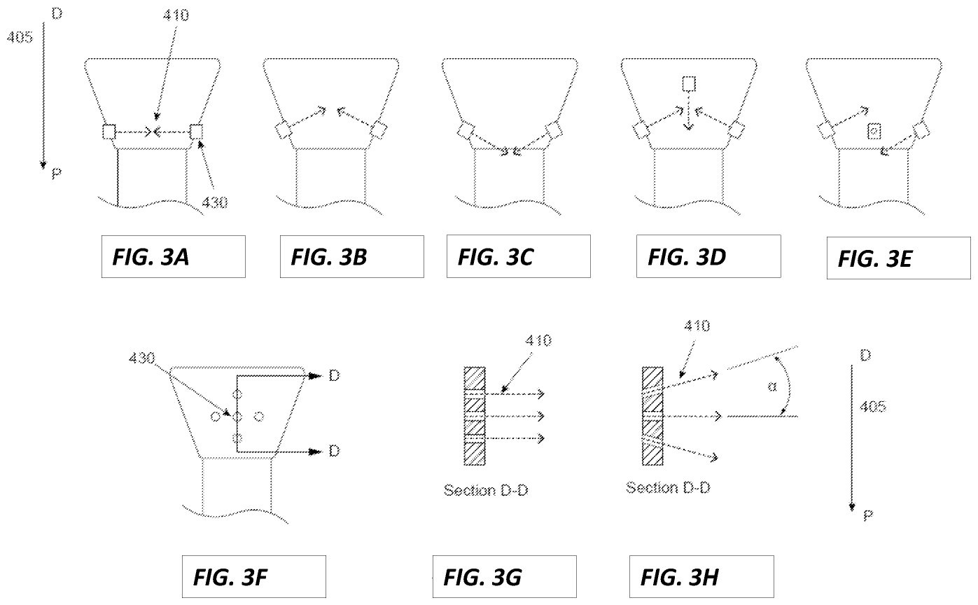

FIGS. 3A-3H depict various configurations of fluid streams 410 that are directed from respective ports 430. A fluid stream 410 can be directed along a path that is substantially orthogonal, proximal, and/or distal to the flow axis 405 (which is like to flow axis 305). In some embodiments, at least two fluid streams are directed in different directions with respect to the flow axis 405. In some embodiments, at least two fluid streams are directed in a same direction (e.g., proximally) with respect to the flow axis 405. In some embodiments, at least a first fluid stream is directed orthogonally, at least a second fluid stream is directed proximally, and at least a third fluid stream is directed distally with respect to the flow axis 405. An angle α may characterize an angle that a fluid stream 410 is directed with respect to an axis that is orthogonal to the flow axis 405 (e.g., as shown in section D-D of FIGS. 3G and 3H). An intersection region of fluid streams can be within an interior portion of the thrombus removal system, and/or exterior (e.g., distal) to the thrombus removal system. In some embodiments, a fluid stream that is directed by a port 430 in a nominal direction (e.g., distally) is deflected along an altered path (e.g., proximally) by (e.g., suction) pressure generated by the aspiration mechanism during operation.

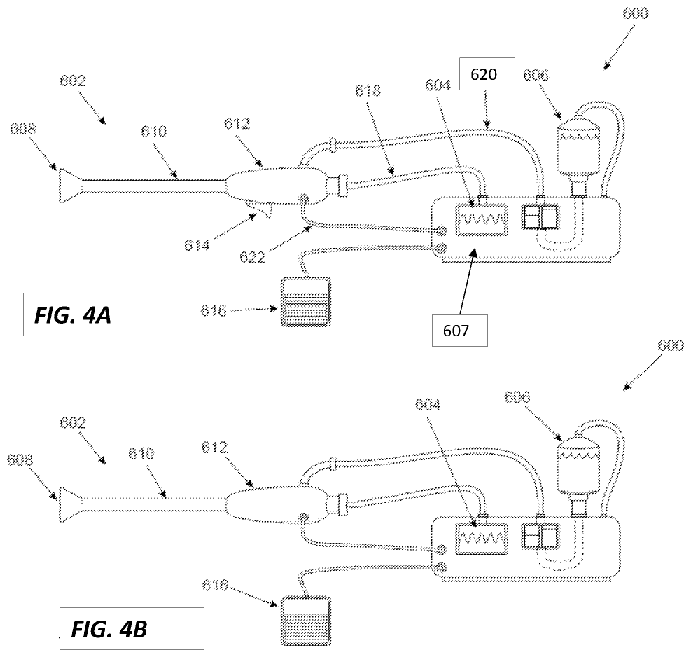

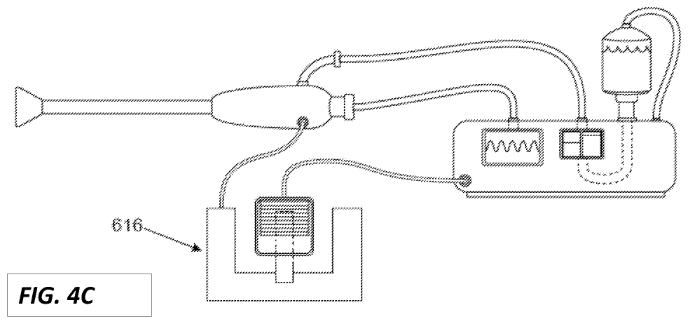

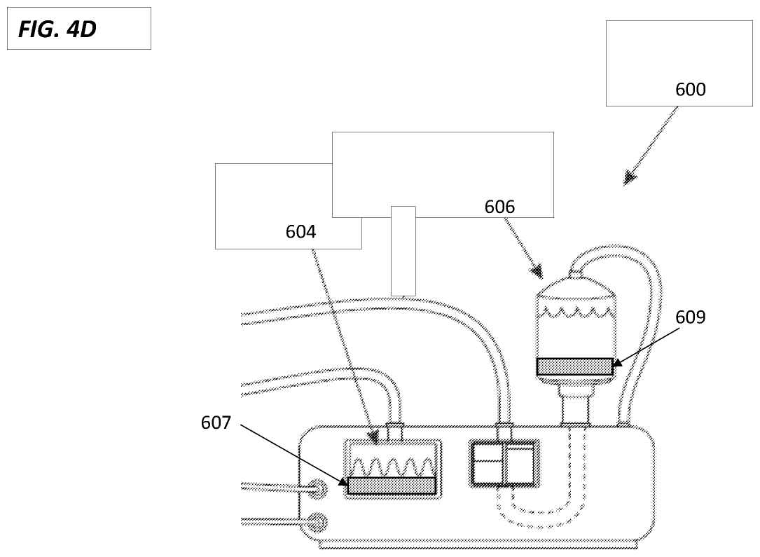

FIGS. 4A-4D illustrate various configurations of a thrombus removal system 600, including a thrombus removal device, 602, a vacuum source and cannister 604, and a fluid source 606. In some embodiments, the vacuum source and cannister and the fluid source are housed in a console unit that is detachably connected to the thrombus removal device. A fluid pump can be housed in the console, or alternatively, in the handle of the device. The console can include one or more CPUs, electronic controllers, or microcontrollers configured to control all functions of the system. The thrombus removal device 602 can include a funnel 608, a flexible shaft 610, a handle 612, and one or more controls 614 and 616. For example, in the embodiment shown in FIG. 4A, the device can include a finger switch or trigger 614 and a foot pedal or switch 616. These can be used to control aspiration and irrigation, respectively. Alternatively, as shown in the embodiment of FIG. 4B, the device can include only a foot switch 614, which can be used to control both functions, or in FIG. 4C, the device can include only an overpedal 616, also used to control both functions. It is also contemplated that an embodiment could include only a finger switch to control both aspiration and irrigation functions. As shown in FIG. 4A, the vacuum source can be coupled to the aspiration lumen of the device with a vacuum line 618. Any clots or other debris removed from a patient during therapy can be stored in the vacuum cannister 604. Similarly, the fluid source (e.g., a saline bag) can be coupled to the fluid lumens of the device with a fluid line 620.

Still referring to FIG. 4A, electronics line 622 can couple any electronics/sensors, etc. from the device to the console/controllers of the system. The system console including the CPUs/electronic controllers can be configured to monitor fluid and pressure levels and adjust them automatically or in real-time as needed. In some embodiments, the CPUs/electronic controllers are configured to control the vacuum and irrigation as well as electromechanically stop and start both systems in response to sensor data, such as pressure data, flow data, etc.

As is described above, aspiration occurs down the central lumen of the device and is provided by a vacuum pump in the console. The vacuum pump can include a container that collects any thrombus or debris removed from the patient.

FIG. 4D is a close-up view of the console of the thrombus removal system, which can include the vacuum source and cannister 604 and the fluid source 606. In some embodiments, the cannister 604 and/or the fluid source 606 can include features designed and configured to assist in determining or estimating therapy progress, including determining or estimating the amount (e.g., volume) or percentage of clot removed. Additionally, the cannister 604 and/or the fluid source 606 can include features designed and configured to assist in determining the amount of fluid (e.g., jets) delivered into the patient and/or the amount of blood removed or aspirated from the patient.

In FIG. 4D, cannister 604 can include sensor 607. In one embodiment, sensor 607 can comprise one or more weight scales configured to measure or sense the weight of fluids and biological materials inside the cannister. The weight scale(s) can be zeroed prior to therapy, and can provide real time weight measurement of the amount of fluid and/or biological materials removed or aspirated from the patient during a clot removal procedure.

In some embodiments, the cannister itself can include a drain with a drain size or filter configured to allow drainage of fluids from the cannister (such as blood and/or saline) while preventing clot material or other biological tissues from draining from the cannister. In this manner, the cannister and weight scale(s) are able to measure only the weight of the clot removed, and not blood and/or saline. Optionally, the blood and other fluids such as saline can be drained into a separate cannister (not shown), which can then be used to determine the amount of blood removed from the patient in addition to the amount of clot removed. In one implementation, fluid source 606 can also include sensor 609, which can be used to track the amount of fluid or saline delivered to the patient through jetting. The sensor 609 can comprise, for example, additional weight scales, or optionally, any other sensor configured to measure the flow of fluid such as a flow sensor. The fluid delivered by the fluid source 606 can be measured by sensor 609 and subtracted from the fluid drained into the separate (not shown) cannister. Therefore, the amount of clot can be determined with weight scales 607 in the cannister 604, and the amount of blood can be calculated in the separate container by subtracting the collected volume from the amount of saline delivered.

In another embodiment, the sensor 607 on or in the cannister 604 can comprise a camera. In some embodiments, the camera can comprise a miniature or fiber optic camera. In some implementations, the camera can be configured to provide real-time imaging of the cannister to provide a visual guide to the user regarding what is being aspirated from the patient. For example, the user can visualize the amount and/or size of clot being removed. The images from the camera can be displayed for the user, such as on a display that provides additional information about the status of the system, device, and procedure.

In other embodiments, the sensor 607 on or in the cannister 604 can comprise other types of sensors, such as optical sensors, flow sensors, etc. Generally, the sensors can be used to monitor or characterize the amount and/or type of material or fluid that enters the cannister to provide the user with additional information regarding the status of the therapy.

Mechanical Manipulation Features for Engagement with Tissue or Material at Distal End of Device (e.g., Grabber Arms)

Some embodiments of a thrombus removal device can include features enabling mechanical manipulation or engagement with tissue or material at a distal end of the device. These features can be referred to herein as mechanical engagement features, grabber arms, fangs, mechanical manipulation arms, mechanical cutting arms, or the like. The grabber arms are generally designed and configured to engage and pull clots into the distal end of the device (e.g., the funnel and/or aspiration lumen). In various implementations, the mechanical engagement features disclosed herein are configured to achieve some combination of pulling thrombus into the funnel, pulling thrombus into the jet plane, pulling thrombus into the aspiration lumen, and/or breaking off or cutting pieces of the thrombus into pieces sufficiently small to be aspirated through the aspiration lumen. While the thrombus removal systems described herein generally include an aspiration lumen and one or more fluid streams or jets, it should be understood that the grabber arms may be implemented in devices without aspiration lumens or without one or more fluid streams or jets. Additionally, the devices described herein generally include an expandable funnel on the distal end of the device. However it should be understood that some embodiments with mechanical engagement features may not include an expandable funnel, but instead some other structure on or near the distal end of the device.

Mechanical engagement features can comprise an arrangement of fangs, arms, or actuatable members positionable at a distal end of a device, such as a thrombus removal device. In some embodiments, the mechanical engagement features are positioned within a distal end of the device (e.g., within a funnel of the device), however other embodiments contemplate positioning the mechanical engagement features outside of the distal end (e.g., funnel), or alternatively, inside an aspiration lumen of the device.

The mechanical engagement features described herein do not include any components that extend distally beyond the distal end of the expandable member or funnel. In general, all actuation or movement of the mechanical engagement features is provided within the confines of the expandable member or funnel. In some embodiments, the mechanical engagement features can include cutting or serrated edges, sharp points, or shearing/pinching mechanisms of action against a targeted clot or tissue. Maintaining the entirety of the mechanical engagement features within the funnel or expandable member increases patient safety and prevents accidentally damaging, cutting, or piercing sensitive tissues such as vessel walls.

The mechanical engagement features described herein can generally include an at-rest state in which the mechanical engagement features are generally not obstructing a central or aspiration lumen of the device (e.g., resting near, adjacent to, or against an inner wall of the expandable member or funnel). The mechanical engagement features can also include an actuated or closed state in which the mechanical engagement features are manipulated to move, either axially and/or radially, towards the central or aspiration lumen of the device. However, in some embodiments, the at rest state includes engagement members that extend into the expandable member or funnel, and an actuated state in which the engagement members are near, rest against, or contact the funnel or expandable member. In some embodiments, this manipulation causes the mechanical engagement features to move axially towards the central or aspiration lumen, and in other embodiments, the manipulation causes the mechanical engagement features to move radially across the expandable member or funnel towards or across a central axis of the opening.

Generally, actuation or manipulation of the mechanical engagement features results in movement along a pivot within the expandable member or funnel. The pivot provides an inflection point between the mechanical engagement feature and the actuating member (e.g., a pull wire, an outer sheath, etc.). While this disclosure discussed movement of the mechanical engagement features as either being axially (e.g., distal to proximal) or radially (e.g., across the funnel or expandable member) it should be understood that since the mechanical engagement features of this disclosure typically move along a pivot, the movement characteristics may be more complex (e.g., a mechanical engagement feature may first swing radially towards a center of the expandable member or funnel before then swinging more axially towards the opening or aspiration lumen of the device).

The mechanical engagement features described herein typically also are directed or face inwards towards a central axis of the device (as opposed to facing outwards towards a vessel wall.

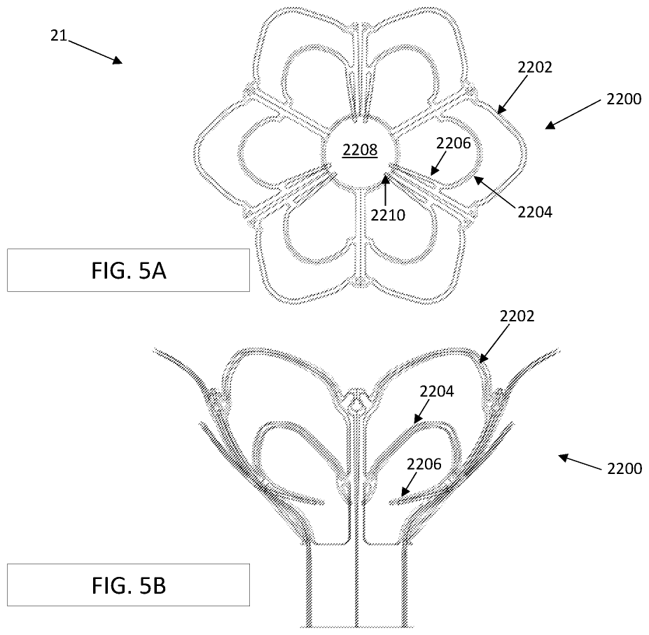

FIGS. 5A-5B illustrate a top-down view and a side-view, respectively, of one embodiment of a distal end 21 of a device which can include additional functionality for delivery and therapy. In the illustrated embodiment, the distal end 21 can include a frame 2200 comprising a plurality of petals that can include an outer frame 2202 and an inner frame 2204. In the illustrated embodiment, the distal end frame is shown having a total of 6 petals, but it should be understood that in other embodiments any number of petals can be implemented, including 2, 3, 4, 5, 6, 7, 8, 9, 10 or more petals. Also, while the petals and petal features are described independently herein, it should be understood that in some embodiments, the frame is a unitary design in which the entire structure, including the plurality of petals, is typically a unitary structure and is manufactured from a single piece of metal (e.g., the entire pattern is laser cut from a piece of nitinol or other appropriate metal or material). The structure of the distal end frame after cutting and shaping can be a single piece of material (e.g., nitinol).

Referring still to FIGS. 5A-5B, the frame can additionally include one or more mechanical engagement features 2206 arranged near or adjacent to an opening 2208 in the distal end. The mechanical engagement features can be generally inwards facing (e.g., facing inwards towards a central axis of the device). In some embodiments of the device (e.g., when the device is a thrombectomy removal device), the opening 2208 can coincide with an aspiration lumen of the device. As will be described in more detail below, the mechanical engagement features can be manipulated or actuated so as to cause distal tips 2210 of the mechanical engagement features 2206 to move or pivot axially towards or away from the opening 2208.

In some implementations, the mechanical engagement features of FIGS. 5A-5B can be manipulated or actuated manually by a user of the device, such as by engaging with pull wires or sliding/rotating an outer sheath over the device. In other embodiments, the actuation or manipulation can be automated such as by coupling the engagement features to motors configured to actuate pull wires or translate/rotate an outer sheath. The motors can be controlled by the user, such as by interacting with a user input device on the device handle or console (e.g., buttons, levers, switches, triggers, etc.).

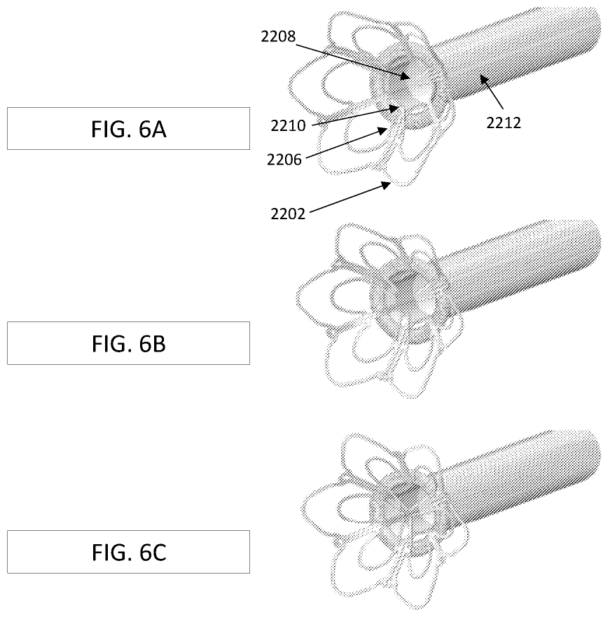

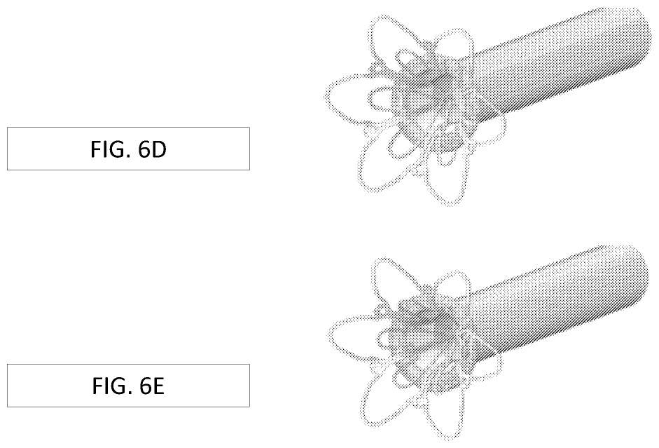

FIGS. 6A-6E illustrate the relative motion of distal tips 2210 of mechanical engagement features 2206 as they pivot towards opening 2208. Referring to FIG. 6A, the funnel or expandable member is fully expanded and in this configuration the mechanical engagement features 2206 are extended fully away from the opening 2208 in the axial direction. In this configuration, the delivery sleeve 2212 is pulled proximally away from the distal end such that it does not provide any forward/distal pressure against the distal end, to allow the distal end to fully expand. Alternatively, the distal end can be pushed out of the delivery sheath.

Referring to FIG. 6B, the delivery sleeve can be advanced slightly so as to apply pressure or contact to a portion of the distal end, including to a portion of the outer frame (2202 in FIG. 5A) and/or inner frame (2204 in FIG. 5A). In some embodiments, the delivery sleeve can be manipulated or advanced manually, such as by a user of the device. In other embodiments, the delivery sleeve can be actuated or manipulated automatically, such as with a motor or other mechanical device. In additional embodiments, the delivery sleeve can be actuated rapidly back and forth along the shaft of the device. Relative movement of the delivery sheath in the distal direction (e.g., against the expanded funnel) causes the inner frame and mechanical engagement feature and distal tip to move or pivot generally proximally in direction towards the opening or aspiration lumen of the device. It should be noted that in this embodiment, the mechanical engagement features have an at rest state in which they are extended outwards and into the funnel or expandable device. Actuating these mechanical engagement features causes them to move proximally towards the opening but also causes them to approach or contact the interior wall of the funnel or expandable member thereby opening or unobstructing the funnel. It is noted that other embodiments described herein include an opposite configuration (i.e., the at rest state leaves the mechanical engagement features out of the way of the funnel, and the actuated state causes the mechanical engagement features to extend into the funnel or expandable member).

Each subsequent drawing, FIGS. 6C, 6D, and 6E, shows the delivery sleeve being advanced slightly more distally over the distal end, causing the distal tip and mechanical engagement features of the distal end to move or pivot proximally towards the opening and/or aspiration lumen of the device. It should be understood that the distal tip design, including the inner and outer frames, allows the mechanical engagement features to be manipulated with the delivery sleeve while maintaining full expansion (or nearly full expansion) of the distal end/funnel. For example, the inner frames can be coupled to the mechanical engagement features, and when the delivery sleeve is advanced over the funnel, the inner frames can be designed to be contacted by the sleeve to move the mechanical engagement features while still allowing the outer frames to maintain full expansion of the device, and therefore full contact with the lumen or vessel. It is noted that the mechanical engagement features of FIGS. 6A-6E do not extend past the distal end of the expandable member or funnel (e.g., past outer frame 2202). The outer frame or funnel/expandable member therefore protects exterior tissues (e.g., vessel walls) from the movement/pivoting and actuation of the mechanical engagement features, allowing them to interact only with the target clot captured by the funnel/expandable member.

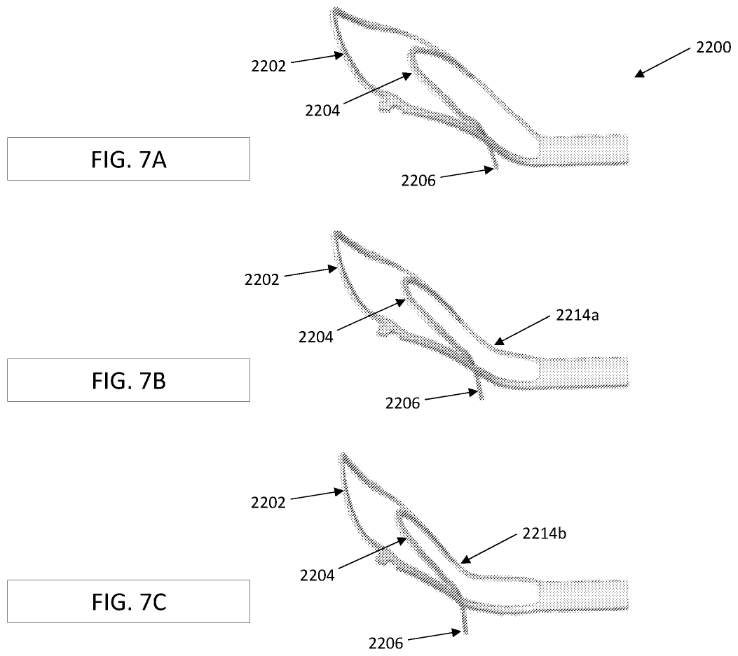

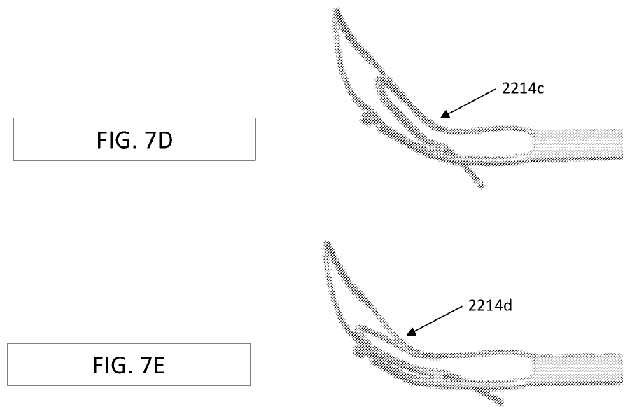

FIGS. 7A-7E illustrate the concept of actuating or manipulating the mechanical engagement features by contacting selected portions of the distal end/funnel/frame with the delivery sleeve. Referring to FIG. 7A, a single petal 2200 is shown in which the distal end is not contacted by the delivery sleeve. In this example, the distal end is allowed to be in its fully expanded configuration, causing the mechanical engagement feature 2206 to move fully axially away from (or distal from) the opening or aspiration lumen of the device (not shown). Referring to FIG. 7B, the delivery sheath (not shown for purposes of description) is advanced distally over the device so as to contact the distal end at location 2214a. In one embodiment, the sheath is configured to contact only the inner frame 2204 of the distal end, thereby causing the mechanical engagement feature 2206 to move or pivot towards the opening or aspiration lumen (or proximally relative to the position of the mechanical engagement feature in FIG. 7A). FIG. 7C shows the delivery sheath being advanced even further distally to location 2214b, causing the mechanical engagement feature to move or pivot even closer to the opening (e.g., axially in the proximal direction relative to the device). Similarly, in FIGS. 7D and 7E, the sheath is further advanced to locations 2214c and 2214d, respectively, causing further pivoting/deflection/movement of the mechanical engagement features. As the sheath is moved past locations 2214a,b,c, and d, the tip of the engagement feature 2206 starts to roll out of the fluid path. This arises in part from the interaction with inner frame 2204. It can be seen in the embodiment of FIG. 7E that the delivery sheath can begin to contact the outer frame, which can cause some contraction or collapse of the distal end. In this embodiment, it may be possible that the distal end collapses enough so as to not fully contact the vessel wall. Typically it is desirable to not collapse the funnel, so care can be taken by the user or system to not advance the sheath to the point where the outer frame is contacted or compressed. In some embodiments, the device can include a stop limiter that is configured to prevent the sheath from collapsing or compressing the expandable member or funnel.

The ability to manipulate the mechanical engagement features provides additional functionality to a medical device such as a thrombectomy removal device during therapy. For example, in some embodiments, the mechanical engagement feature(s) can be designed and configured to engage with a clot that is in the funnel. In some embodiments, this physical or mechanical interaction with the clot can be leveraged so as to physically pull or move the clot into contact with the device. Depending on the configuration of the device (e.g., funnel, aspiration lumen, one or more jets, etc.), the mechanical engagement features can be used to 1) pull the clot into contact with jets or into a plane of the jets, to thereby break up the clot and aspirate the clot, 2) pull the clot into or towards the aspiration lumen, and/or 3) prevent the clot from exiting the distal end or funnel of the device. The combination of the mechanical engagement features, the jets, and aspiration allow for clot removal capabilities not previously enabled by other devices. The combination can also cut or help cut the clot while pushing it into the aspiration lumen or jet plane.

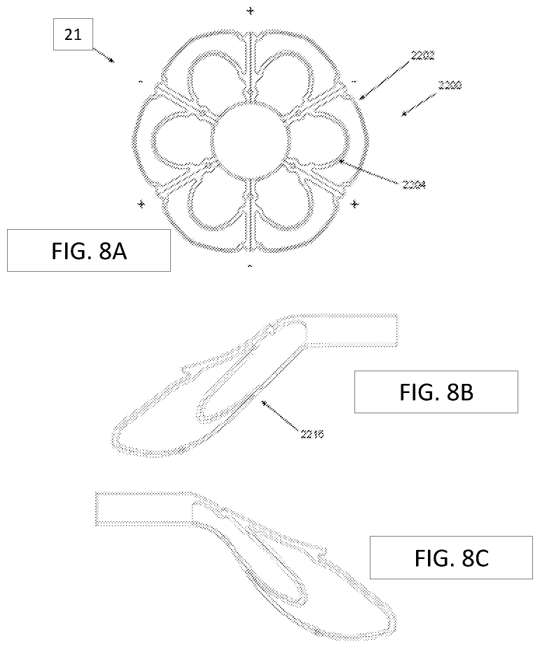

FIGS. 8A-8C illustrate another embodiment of a distal end 21. This distal end frame design can still include a petal shaped frame 2200 including outer frames 2202, and inner frames 2204. While this embodiment is illustrated without the previously described mechanical engagement features, it should be understood that variants can include one or more mechanical engagement features. However, referring to FIGS. 8B and 8C, alternating frame petals of the distal end can include varying side profiles to customize the way in which the funnel interacts with a delivery sheath. For example, referring to FIG. 8B, It can be seen that the outermost side profile of the outer frame and inner frame, represented by reference number 2216, shows a generally linear or straight profile. In contrast, the outermost side profile of the outer frame and inner frame in FIG. 8C, represented by reference number 2218, includes a slightly curved or bowed-out profile. For purposes of illustration, referring back to FIG. 8A, petal or frame edges having the flat profile shown in FIG. 8B can be represented by the (−) symbol, and petal edges having the curved or bowed-out profile shown in FIG. 8C can be represented by the (+) symbol. It can be seen in FIG. 8A that alternating petal edges can have alternating (+) and (−) side profiles. In doing so, the work required to advance the delivery sheath over the funnel can be reduced. More specifically, if half the petal edges have the (+) profile and half the petal edges have the (−) profile, advancement of the delivery sheath will only create contact with the (+) profile edges, thereby reducing friction between the funnel and sheath (e.g., contact with only 3 petal edges instead of 6). Additionally, the (−) profile petal edges can still be designed and configured to absorb some of the deformation caused by advancement of the sheath, further reducing the forces required.

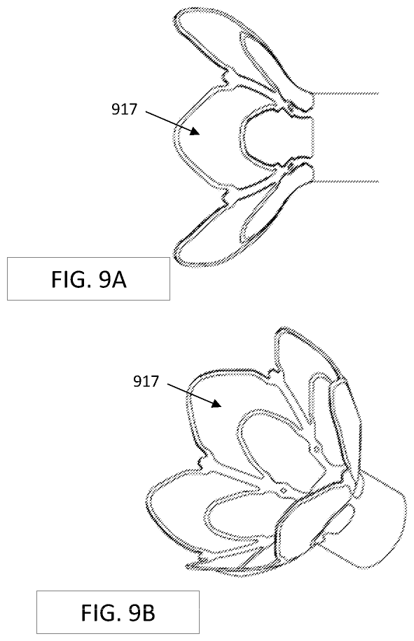

The distal end embodiment of FIGS. 9A-9B can include similar structure to that as described above. However, in this embodiment the distal end, including the inner and outer frames, can include a membrane 917 or other covering such as an elastomer covering (e.g., a thermoplastic urethane, or silicone) or other membrane material as known in the art. In some embodiments, the membrane 917 can fill in the interior portions of the frame, including those surrounded by the inner and outer frames. In other embodiments, the membrane can cover the entirety of the frame.

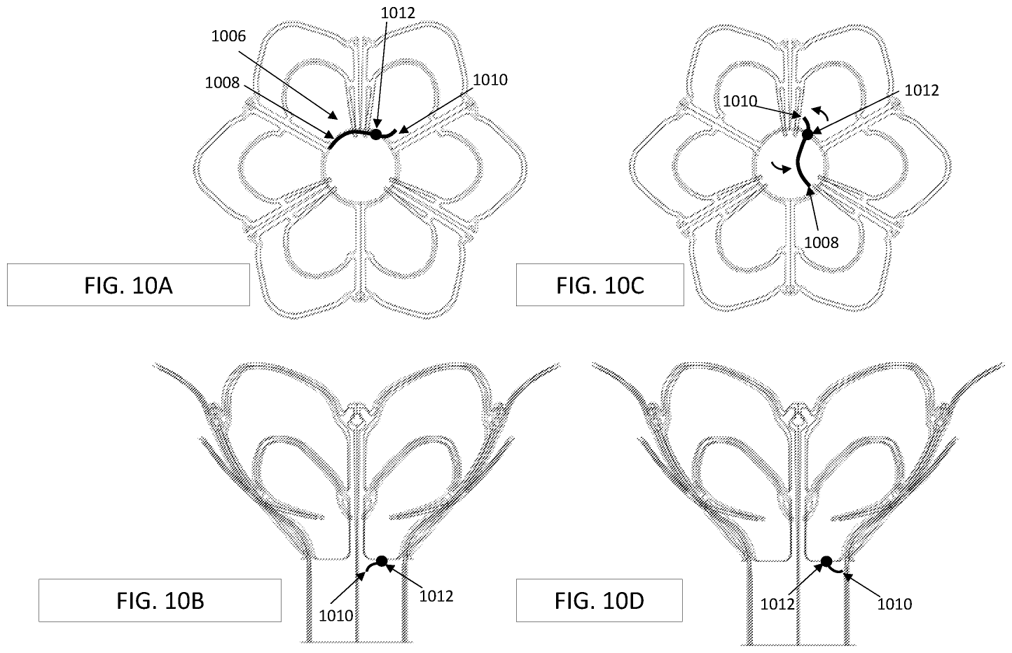

Referring to FIGS. 10A-10D, various alternative embodiments of mechanical engagement features 1006 that may be actuated manually or automatically (e.g., with a motor or other automated actuation source) are depicted. As shown in FIGS. 10A-10B, the mechanical engagement feature 1006 includes a distal portion 1008, a proximal portion 1010, and a hinge 1012 adapted to rotate and/or pivot the distal portion 1008 from a first, at-rest position to a second, actuated position. The actuation of the mechanical engagement feature can be generally in a radial direction, that is, within a given axial position within the distal end and/or catheter body (e.g., FIG. 10B). In some embodiments the mechanical engagement feature, when actuated, functions as a cutter (e.g., blade or knife), cutting into portions of any captured clot. In some embodiments, the distal portion 1008 may be sharp or serrated to improve cutting ability. Actuation of the mechanical engagement feature can be made by advancement or rotation of an outer catheter sheath, a pull wire, or any other actuation approaches described herein. FIGS. 10A-10B illustrate the mechanical engagement feature in an at rest configuration in which the distal portion 1008 is near or adjacent to the frame of the distal end or funnel, so as to not occlude or interfere with a central opening or lumen (e.g., aspiration lumen) of the distal end. In this illustrated embodiment, referring to FIG. 10C, the proximal portion 1010 can be rotated or actuated, such as with an external sheath, to cause the inward-facing distal portion 1008 to rotate across the distal portion (e.g., across the aspiration lumen). Movement about the pivot of the mechanical engagement feature is indicated with the arrows in FIGS. 10C-10D. Although only one such mechanical engagement feature is shown in the embodiment of FIGS. 10A-10D, it should be understood that any number of engagement features can be implemented (e.g., 2, 3, 4, or more mechanical engagement features within the expandable member or funnel). It is also noted that while the features shown in the embodiment of FIGS. 10A-10B include a proximal portion that is actuated with an external sheath, other embodiments are contemplated in which there is no proximal portion, only the inward-facing distal portion 1008, and actuation can be with other mechanisms including, for example, pull wires.

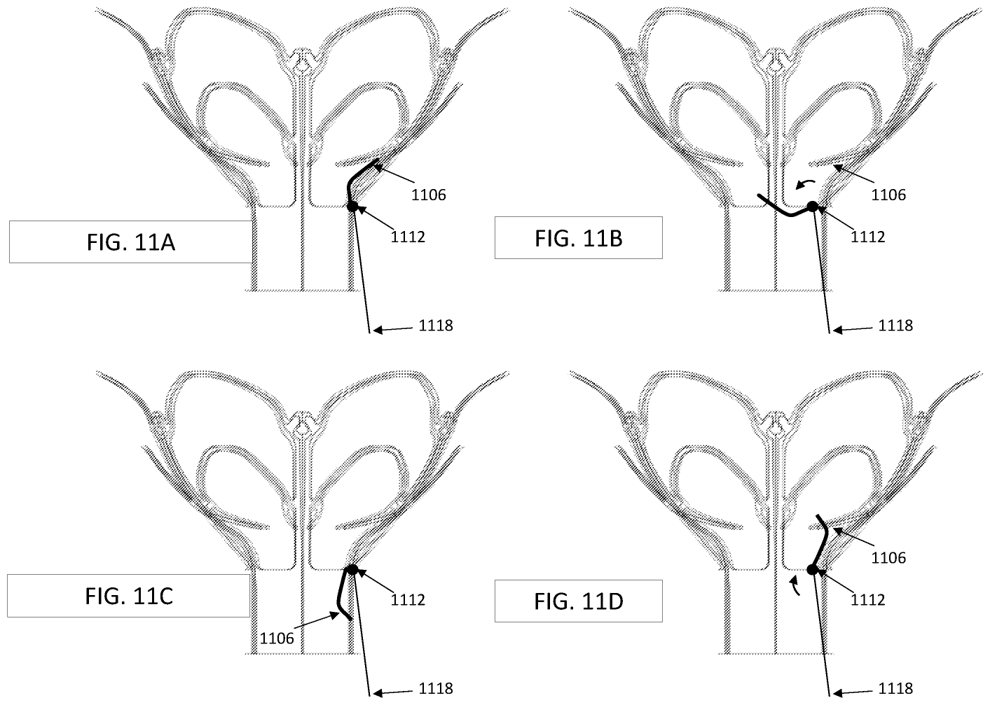

Referring to FIGS. 11A-11D, variations of the mechanical engagement features of FIGS. 10A-10D, but with elements that are disposed to be radially inward-facing, and actuated via a hinge or pivot region to move in a proximal or distal direction. As shown, actuation of the mechanical engagement feature(s) 1106 can be accomplished with a pull-wire 1118. In another embodiment, actuation can include advancement of an outer catheter over an external portion of the actuation element (as in the embodiment of FIGS. 10A-10D), causing the hinge to pivot the internal region in the proximal direction. In the embodiment of FIGS. 11A-11B, the mechanical engagement member can have an at rest configuration as shown in FIG. 11A, with the mechanical engagement member resting against the distal end, frame, or funnel distally to the pivot 1112. Actuation then causes the mechanical engagement feature to swing down or proximally towards the lumen or aspiration lumen of the device, as indicated by the arrow. Alternatively, in the embodiment of FIGS. 11C-11D, the mechanical engagement member 1106 can have an at rest configuration in which the mechanical engagement member is proximal to the pivot 1112, this time within the lumen or aspiration lumen of the device. Actuation then causes the mechanical engagement feature to swing up or distally towards the distal end or funnel of the device, as indicated by the arrow. As with the embodiments described above, the mechanical engagement members can be actuated to pivot either manually or automatically, such as by manipulating an external sheath, manipulating pull wires, or using a motor or other automated feature to manipulate the sheath or pull wires.

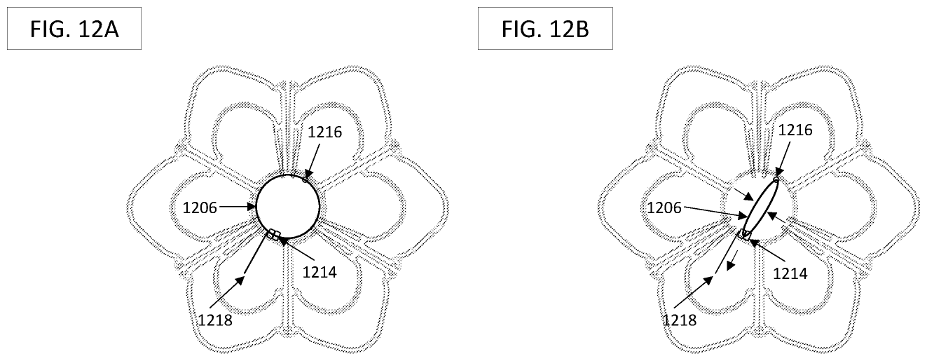

Referring to FIGS. 12A-12B, another variation on a mechanical engagement feature 1106 includes a wire structure that is adapted to act as a lasso or noose. In this embodiment, the mechanical engagement feature 1206 may include a noose-like structure 1214, an anchor 1216, and a pull-wire or actuator 1218. When the pull-wire is pulled, a section of the mechanical engagement feature is held in place by the anchor 1216 while some of the mechanical engagement feature is allowed to slip through the noose-like structure 1214 causing the mechanical engagement feature to collapse upon itself like a slip-knot, as shown in FIG. 12B. The mechanical engagement feature may have a first configuration that is generally open, as shown in FIG. 12A, and a second, actuated configuration that is closed with respect to the first configuration. The second configuration can include movement of the lasso across the center or lumen of the catheter and/or a portion of the distal end. Furthermore, movement of the mechanical engagement feature across the distal end can cut be configured to cut into and fragment captured clot or tissue. The lasso actuator may be actuated by a pull-wire 1218, or actuator. The lasso can reversibly transition from the first configuration to the second configuration. The lasso and/or pull line can be of a Nitinol construction. In some embodiments, the lasso has a shapeset configuration that corresponds to the first (open) configuration, and following actuation, the lasso tends to return to the open configuration.

As described above, several embodiments of actuation for the mechanical engagement features can be implemented. In one embodiment, an outer sheath is cooperatively coupled with the medical device such that relative movement therebetween (e.g., advancement and/or rotation) causes actuation of the mechanical engagement features. In one embodiment, retraction (proximal movement) of an interference element such as a pull-wire or similar actuation element causes the mechanical engagement feature to pivot and/or rotate about a hinge.

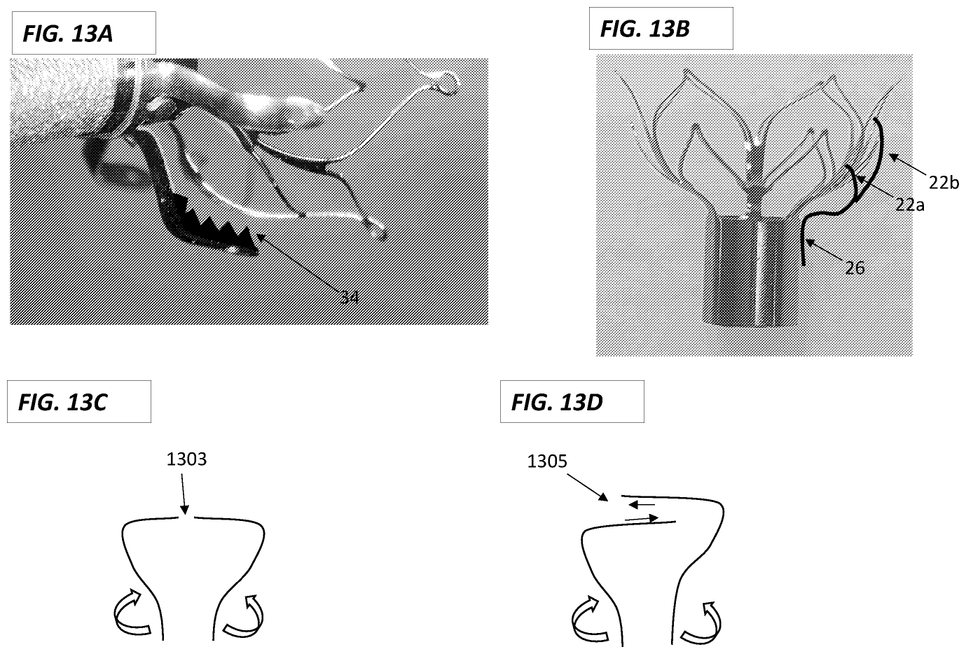

Additional embodiments of mechanical engagement features are shown in FIGS. 13A-13D. In the example of FIG. 13A, one or more of the mechanical engagement features can include a serrated or cutting edge 34. This serrated or cutting edge can be designed and configured to assist with cutting or macerating the clot(s) or tissue at the device distal end when the mechanical engagement features are actuated. This cutting or serrated edge can be along an entire length of the mechanical engagement features or along only a portion of the length of the mechanical engagement features (e.g., along only a distal portion).

FIG. 13B illustrates another example of mechanical engagement features that includes more than one arm or engagement structure for each mechanical engagement feature. In FIG. 13B, actuation of mechanical engagement feature 26 can cause both arms 22a and 22b to be actuated inwards towards the center or aspiration lumen of the device. It should be understood that some examples can include two, three, four, or more arms for each mechanical engagement feature. In the illustrated example, the plurality of arms for each mechanical engagement feature allows the mechanical engagement features to operate at a plurality of heights within the distal end or funnel of the device, potentially increasing the cutting or clot engagement capabilities of the mechanical engagement feature and allowing for engagement with the clot at multiple levels or heights within the funnel.

FIGS. 13C-13D illustrate embodiments in which the mechanical engagement features within the distal end or funnel of the device can be offset or arranged in different ways so as to enhance or change the mechanical interaction between the mechanical engagement features and the clot(s) or tissue. For example, in FIG. 13C, the mechanical engagement features can be designed and configured to collide so as to pinch one or more clots within the funnel at pinch point 1303. In this example, mechanical engagement features on opposite sides of the funnel or distal end are shown, with the distal tips of the mechanical engagement features being designed to contact each other when actuated. In one embodiment, the mechanical engagement features can be arranged so that they first collide and pinch clot material before then pulling the clot in towards the aspiration lumen as actuation of the mechanical engagement features continues. It should be understood that in embodiments where there are more than two mechanical engagement features, not all the mechanical engagement features must be designed to collide and pinch the clot. In some examples, only two of the mechanical engagement features can be arranged in this manner, and the other mechanical engagement features can operate similar to the other mechanical engagement features embodiments described herein. However, in some embodiments, all the mechanical engagement features can be designed and configured to collide at a single point (e.g., at the pinch point).

In the embodiment of FIG. 13D, the distal ends of two or more mechanical engagement features can be offset to create a shearing action 1305 (e.g., like scissors) when the mechanical engagement features are actuated. As with the embodiment of FIG. 13C, embodiments are provided in which two or more mechanical engagement feature distal ends are offset. But it should be understood that not all mechanical engagement features need to be offset, and some mechanical engagement features can operate similar to the other mechanical engagement features embodiments above where they simply actuate to pull clot material inwards towards the aspiration lumen.

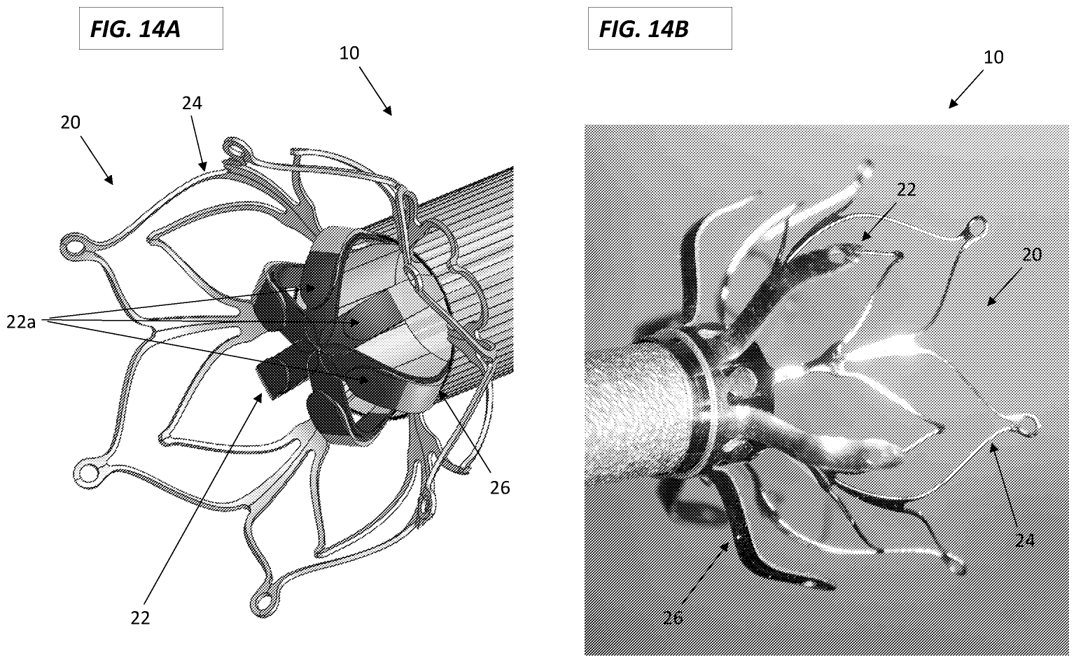

FIGS. 14A-14B illustrate one embodiment of a nested frame approach for a funnel or distal end 20 and mechanical engagement feature array 22 of a medical device 10. As shown in FIGS. 14A-14B, the funnel 20 can include a funnel frame structure 24 that is distally disposed relative to the mechanical engagement features frame structure 26. In some embodiments, the funnel frame structure is independent from or decoupled from the mechanical engagement features frame structure 26. The funnel frame structure 24 can provide radial stiffness and support for the funnel 20. In some embodiments, the funnel frame structure 24 can comprise a shape memory material (e.g., Nitinol) to facilitate automatic expansion of the funnel (e.g., when a sheath or covering of the funnel is removed). The engagement feature frame structure 26 can be configured to actuate the mechanical engagement feature array to cause individual engagement features to grab or engage clots and pull those clots proximally into the funnel and/or towards the aspiration lumen and/or jets of the device. In one embodiment, a sheath (not shown, but previously described in other embodiments) of the device is configured to be moved distally over the shaft of the medical device to engage the mechanical engagement feature frame structure 26 without engaging the funnel frame structure, causing one or more mechanical engagement features of the mechanical engagement feature array 22 to pivot or move about an axis within the expandable member or funnel. The nested frame structure that positions the engagement feature frame structure proximally relative to the funnel frame structure allows for actuation and movement of the mechanical engagement features without collapsing the funnel or distal end.

The mechanical engagement feature array 22 illustrated in FIG. 14A shows an example of an array having a plurality of mechanical engagement features 22a arranged circumferentially around or within the distal end of the medical device 10. Actuation of the mechanical engagement features 22a can advance the distal tips of each mechanical engagement feature within the array towards a central axis of the medical device to grab, manipulate, cut, macerate, or otherwise physically engage the thrombus material. In some embodiments, the mechanical engagement features can be configured to contact, pinch, or shear past another in the central axis of the device, and in other embodiments the mechanical engagement features are short enough to leave an open aperture in the central axis of the device even when actuated or closed. Although a single layer array is illustrated in FIGS. 14A and 14B, other embodiments can include a mechanical engagement feature array comprising one or more levels or layers of mechanical engagement features (e.g., as in the embodiment of FIG. 13B). Each layer of mechanical engagement features may be selectively actuatable independent from other layers to enable selectively engaging the thrombus material depending on the thrombus location within the funnel or distal end.

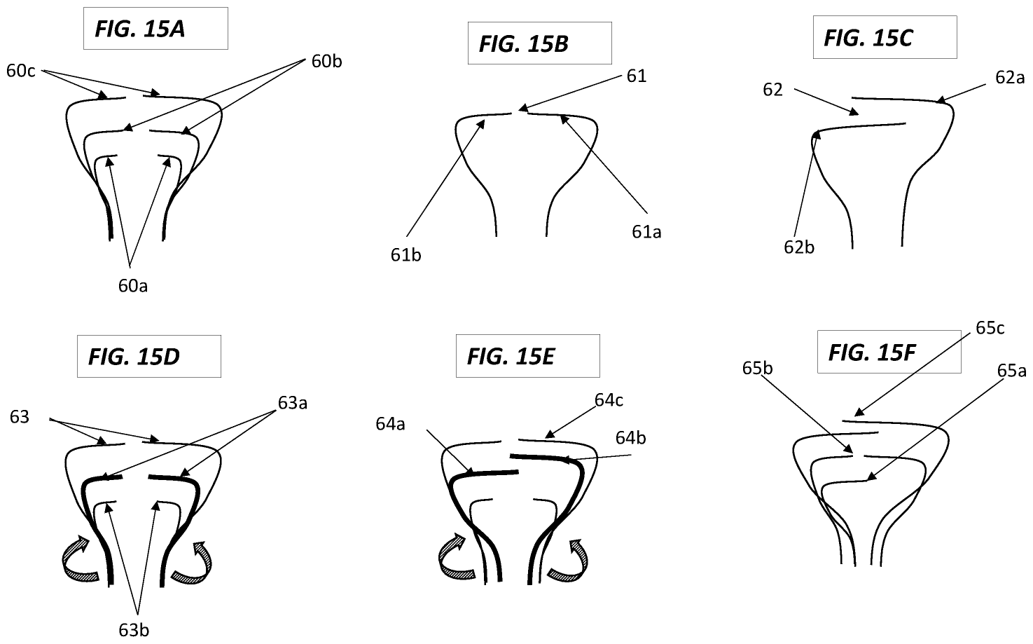

FIGS. 15A-15F illustrate additional examples of mechanical engagement feature arrays from a cross sectional view showing examples of different mechanical engagement feature layers and various examples of engagement configurations. The mechanical engagement features of FIGS. 15A-15F are shown in isolation for purposes of illustration, but it should be understood that they can be disposed entirely within an expandable member or funnel (not shown) as with previously described embodiments. Additionally, these mechanical engagement features can include actuation mechanisms coupled to the engagement features (e.g., pull wires, external sheaths, motors, etc.) to cause the mechanical engagement members to pivot or move within the expandable member/funnel during actuation.

Referring to FIG. 15A, a mechanical engagement feature array is shown comprising three layers of mechanical engagement feature 60a, 60b, 60c axially displaced from another. Mechanical engagement feature 60c may be a distal layer of mechanical engagement features that can be actuated towards one another and a central point (e.g., pinch point) to grab, cut or otherwise engage thrombus material. Mechanical engagement features 60b and 60a may be actuated in combination with mechanical engagement features 60c, independent of 60c, or a combination where one or more mechanical engagement features of each layer 60c, 60b, and 60a are actuatable. The different layers of mechanical engagement features may be configured to interact with the thrombus material in different ways. In some examples, some mechanical engagement features or layers may be configured to retain thrombus material within the medical device (e.g., the funnel or distal end) while other mechanical engagement features or layers are configured to cut, pinch, pull, twist, or rotate thrombus or tissue material. For example, mechanical engagement features 60c may be configured to close towards one another to hold the thrombus material or tissue within the funnel, or prevent the thrombus material or tissue from exiting the funnel, while mechanical engagement features 60b and/or 60a may be actuated to cut, pinch, pull, twist, or rotate the thrombus material.

In some examples, one of more layers of mechanical engagement features may be actuated independent of other mechanical engagement features layers. In some examples, one or more layers of mechanical engagement features may be actuated in combination with one or more additional mechanical engagement features layers. For example, referring to FIG. 15A, one or more mechanical engagement features or mechanical engagement features layers may be actuated independent of one another such as mechanical engagement features 60a being retracted proximally after engaging thrombus material while mechanical engagement features layers 60b and 60c can remain statically engaged to the thrombus material to allow mechanical engagement features layer 60a to cut, tear, or otherwise separate proximal segments of the thrombus material therein. In some examples, one or more mechanical engagement features layers may work in concert with one another to manipulate the thrombus material.

Again referring to FIG. 15A, mechanical engagement features layers 60a, 60b, and 60c may be actuated in series or any other sequence relative to one another. For example, the thrombus removal device may engage thrombus material and one or more mechanical engagement features layers may be actuated to engage the thrombus material. For example, mechanical engagement features layer 60a may engage the proximal segment of thrombus material, then mechanical engagement features layer 60b may subsequently engage the thrombus material following by subsequent distal mechanical engagement features layers (e.g., mechanical engagement features layer 60c). In some examples, the sequence of engagement may be configured to pull or displace the entire thrombus proximally within the thrombus removal device or towards a cutting plane of jets of the device. For example, mechanical engagement features layer 60a may engage the proximal segment of thrombus material and be retracted proximally while mechanical engagement features layer 60b is actuated to engage the thrombus material and support a proximal displacement of the thrombus material followed by subsequent distal mechanical engagement features layers engaging the thrombus and retracting proximally to pull the entire thrombus proximally towards the thrombus removal device (e.g., aspiration catheter).

FIGS. 15A and 15B illustrate examples of how one or more mechanical engagement features layers may engage a thrombus. For example, FIG. 15B illustrates mechanical engagement features 61a and 61b (e.g., opposing mechanical engagement features of the same layer) being designed and configured to collide so as to pinch one or more clots within the funnel or distal end. In this example, mechanical engagement features on opposite sides of the funnel are shown, with the distal tips of the mechanical engagement features being designed to contact each other when actuated. In one embodiment, the mechanical engagement features can be arranged so that they first collide and pinch clot material before then pulling the clot in towards the aspiration lumen as actuation of the mechanical engagement features continues. It should be understood that in embodiments where there are more than two mechanical engagement features, not all the mechanical engagement features must be designed to collide and pinch the clot. In some examples, only two of the mechanical engagement features can be arranged in this manner, and the other mechanical engagement features can operate similar to the other mechanical engagement features embodiments described herein. However, in some embodiments, all the mechanical engagement features can be designed and configured to collide at a single point (e.g., at a central point in the funnel or distal end).

FIG. 15C illustrates another example of how one or more mechanical engagement features layers may engage a thrombus. In this example, the mechanical engagement feature tips of two or more mechanical engagement features can be offset to create a shearing action (e.g., like scissors) when the mechanical engagement features are actuated. In some examples, offset configuration in this way may be configured to shear a segment of the thrombus. In some examples, an offset configuration may be configured to manipulate or displace thrombus material for engagement with one or more mechanical engagement features layers. For example, mechanical engagement features 62b and 62a may be configured to engage different areas of a thrombus to increase the engagement and hold the thrombus in a particular orientation while one or more additional mechanical engagement features layers impact the thrombus material proximally or distally from mechanical engagement features layer 62.

In some examples, a mechanical engagement features layer may comprise a plurality of offset mechanical engagement features configured to be actuated independent or in combination with one another to engage a thrombus and retain the thrombus material in a static position while one or more mechanical engagement features layers can be actuated to impact the thrombus (e.g., shear, cut, macerate, etc.). In some examples, one or more mechanical engagement features of a layer may be configured to engage a thrombus to rotate the thrombus material. As described herein, the closure of mechanical engagement features within a layer or array may be configured to engage the thrombus material and provide rotational force to the material by closing similar to an iris around an aperture.

FIG. 15D illustrates a mechanical engagement features array configuration allowing one or more mechanical engagement features or layers to incorporate apertures and fluid lumens to deliver fluid streams from mechanical engagement features 63a. The fluid delivery mechanism can provide a plurality of fluid streams (e.g., jets) to fluid apertures of the thrombus removal system through one or more apertures within the mechanical engagement features array. For example, one or more fluid streams delivered by the mechanical engagement features may be configured for macerating, cutting, fragmenting, pulverizing and/or urging thrombus to be removed from a proximal portion of the thrombus removal system. Mechanical engagement features 63b and 63 may be configured to retain, cut, twist, slice, pinch, or rotate thrombus material while the mechanical engagement features 63a may be configured to provide fluid streams or jets that contact the thrombus material sufficient to break the segment of thrombus at or near the mechanical engagement features 63a. Resulting proximal segments of thrombus material may be retracted by mechanical engagement features 63b while layer 63 can retain the proximal segment of thrombus material to prevent or reduce potential distal migration of thrombus material. In some examples, a series of cutting of thrombus material by mechanical engagement features layers may be facilitated by the proximal and distal retention of the thrombus material while one or more medial mechanical engagement features layers impact the thrombus material to shear or otherwise break the thrombus allowing the proximal layers (e.g., 63b) to retract the proximal thrombus material segment into the thrombus removal device.

Similar to FIG. 15D, FIG. 15E illustrates an example configuration of mechanical engagement features 64a and 64b incorporating apertures and fluid streams being offset. As described herein, the jets from mechanical engagement features 64a and 64b may be configured to shear or otherwise break apart thrombus material. Distal mechanical engagement features layer 64c may be configured to retain and enclose the thrombus material within the distal end of the thrombus removal device while the fluid streams from mechanical engagement features 64a and 64b impact the thrombus material. For example, distal mechanical engagement features layer 64c may be configured to reduce thrombus fragments from exiting the array while medial and proximal layers engage and break apart the thrombus material.

In some examples, one or more mechanical engagement features or layers may comprise different characteristics configured to impact the thrombus material in a variety of ways. For example, some mechanical engagement features may be more stiff than others. Increased stiffness may provide improved cutting, macerating, or engagement with thrombus material. In some examples, mechanical engagement features with increased stiffness may be configured to cut, macerate, or otherwise deform the thrombus material while more flexible mechanical engagement features can be configured to hold, retain, or otherwise manipulate the thrombus material in support of the stiffer cutting members. FIG. 15F illustrates an example where mechanical engagement feature 65a or any other feature may have increased stiffness, rigidity, or geometry to provide a cutting impact on the thrombus material. Mechanical engagement features 65b may be sufficiently stiff to retain the thrombus material while mechanical engagement features 65a can be configured to impact the thrombus material to cut, tear, macerate, or otherwise deform the thrombus material at a proximal end of the array for improved aspiration of pieces of the thrombus material separated by mechanical engagement feature 65b.

Additionally, FIG. 15F illustrates an example of increased articulation of mechanical engagement features relative to one another. For example, the space between mechanical engagement features 65b may illustrate a pinch point generally central or towards a central axis of the thrombus removal device, while the distal layer 65c is illustrated as overlapping mechanical engagement features that can be configured to engage the thrombus at more than one point or area increasing the engagement with the thrombus material to prevent distal migration or inadvertent separation of a distal segment of thrombus material. Mechanical engagement features 65a may be a single mechanical engagement feature or a single actuated mechanical engagement feature from a layer that can be actuated to pull against the proximal portion of thrombus material. For example, mechanical engagement features 65a may be actuated to engage the thrombus at a proximal segment of the thrombus material and the mechanical engagement feature may be retractable while layers 65b and 65c maintain a static engagement with the thrombus material thereby allowing mechanical engagement feature 65a to cut, tear, or otherwise separate thrombus material proximally into the thrombus removal device.

In some embodiments, the stiffness of the mechanical engagement feature frame structure can be tuned independently of the stiffness of the funnel frame structure. For example, it may be desirable to have a funnel that is as compliant as possible so as to avoid injuring or damaging delicate vessel structures. At the same time, it may also be desirable to have a mechanical engagement feature frame structure and mechanical engagement features that are more stiff than the funnel, to provide improved clot engagement or maceration. Alternatively, it may be desirable to have the mechanical engagement features be more compliant than the funnel itself. Regardless, in some embodiments, the mechanical engagement features frame structure is stiffer than the funnel frame structure, and in other embodiments, the mechanical engagement features frame structure is less stiff than the funnel frame structure. Alternatively, the mechanical engagement features frame structure can have substantially the same stiffness as the funnel frame structure.

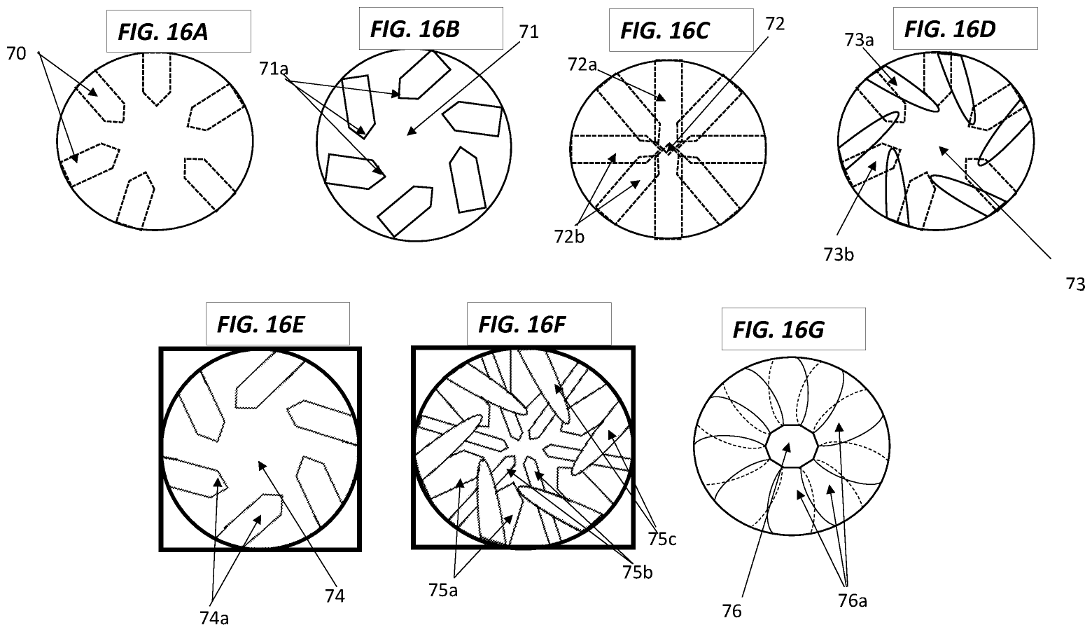

FIGS. 16A to 16G illustrate cross-sectional views of mechanical engagement features arrays disposed within an expandable member or funnel, as viewed from the distal end of the medical device. The layers of mechanical engagement features within an array may be configured to actuate and extend outward, inward, laterally, diagonally, orthogonally, etc. or a combination thereof to engage a thrombus or otherwise impact thrombus material within the distal end of the thrombus removal device. Referring to FIG. 16A, one or more layers may comprise a plurality of mechanical engagement features 70 configured to actuate and enclose towards or beyond a central point (e.g., a central axis). In some examples, the mechanical engagement features 70 can be actuated or controlled independently, in groups, all-together, or some combination thereof.

In FIG. 16B, another example of a layer of mechanical engagement features 71a is shown closing in a cyclone or cylindrical manner around a central axis 71. The mechanical engagement features 71a may be arranged in a single layer or offset relative to one another. The cylindrical closing of mechanical engagement features 71a can be similar to an iris closing around a central aperture and may be controllable via mechanical engagement feature articulation to increase or decrease the aperture provided around the central axis 71. In some examples, the mechanical engagement features may be configured to close entirely to retain or sever thrombus material therein. In some examples, mechanical engagement features 71a may be configured to close in sequence relative to one another to rotate the thrombus material retained therein. In other examples, the mechanical engagement features 71a can be actuated or controlled independently, in groups, all-together, or some combination thereof.

In FIG. 16C, a layer may comprise one or more teeth having different dimensions or level of articulation. For example, mechanical engagement features 72a may be longer or extend further towards one another while mechanical engagement features 72b may extend to the central axis 72 or to some point before the central axis. For example, mechanical engagement features 72a may be configured to shear the thrombus material while mechanical engagement features 72b provide lateral support of the thrombus material being sheared. In some examples, the mechanical engagement features 72a and 72b can be actuated or controlled independently, in groups, all-together, or some combination thereof.

FIG. 16D, similar to FIG. 16B illustrates an example of one or more mechanical engagement features or layers 73a/73b configured to engage a thrombus material when actuated to close around the central aperture 73 in a cylindrical or cyclone manner. Here, multiple layers of mechanical engagement features are shown closing in a concentric cyclone. However, one or more layers may be actuated to enclose around the central aperture in a clockwise direction and one or more layers may be configured to close in a counterclockwise direction. In some examples, all layers may be configured to close or otherwise adjust the central aperture in a clockwise direction. In some examples, all layers may be configured to close or otherwise adjust the central aperture in a counterclockwise direction. In some examples, one or more layers may close or otherwise adjust the aperture size in a clockwise direction while one or more additional layers may close or adjust the aperture in a counterclockwise direction. For example, one or more layers may be configured to twist thrombus material therein. In some examples, the mechanical engagement features 73a/73b can be actuated or controlled independently, in groups, all-together, or some combination thereof.

In some examples, shown in FIG. 16E, the dimensions of mechanical engagement features 74a may be configured to engage the thrombus material to retain the thrombus without shearing the thrombus. Mechanical engagement features 74a are positioned at a distance from one another around the circumference of the distal end of the thrombus removal device to pierce or engage the thrombus material without shearing. In some examples, one or more layers may be configured to pierce and retain the thrombus material. In some examples, one or more layers may be configured to engage or apply pressure to the thrombus without piercing the thrombus material. In some examples, engaging the thrombus material in this way can provide support for one or more additional layers to shear, separate, cut, macerate, or otherwise segment thrombus material. In some examples, the mechanical engagement features 74a can be actuated or controlled independently, in groups, all-together, or some combination thereof.

In some examples, the thrombus removal device may comprise an array having any number of mechanical engagement features layers. Each layer may be configured to manipulate the thrombus material therein as described herein. For example, a proximal layer may be configured to shear proximal thrombus segments, distal layers may also be configured to shear, retain, apply fluid pressure, enclose, rotate, macerate, etc. or a combination thereof to different segments of thrombus material therein.

FIG. 16F shows another example of one or more layers of mechanical engagement features within a mechanical engagement features array having different geometry, characteristics, and associated function. Layer 75c may be a distal layer configured to be actuated and close in a cyclone manner while proximal layers 75a and 75b can be configured to close in a more linear manner around the central axis. In some examples, the proximal layers may be configured to retain the thrombus material at a proximal segment while one or more distal layers shear, cut or separate the thrombus material from a distal portion outside of the thrombus removal device. In some examples, the mechanical engagement features 75a-75c can be actuated or controlled independently, in groups, all-together, or some combination thereof.

In some examples, any layer may incorporate apertures and fluid lumens to provide one or more jets, as described herein. For example, referring to FIG. 16F, layer 75b may be configured to produce one or more jets or fluid streams to separate or otherwise detach a proximal segment of thrombus material that may be aspirated or retracted proximally within the thrombus removal device.

FIG. 16G illustrates yet another example of a layer having overlapping mechanical engagement features 76a that can be configured to close laterally or cyclonically around the central lumen (e.g., aperture) 76. In this example, the mechanical engagement features may be configured to slice the thrombus at the central aperture 76. In some examples, a layer configured in this manner may be a distal layer configured to enclose all or part of a thrombus material within the distal end of the thrombus removal device. For example, a thrombus may be engaged by the distal end of the thrombus removal device and the distal layer (e.g., mechanical engagement features 76a) may be actuated to enclose the thrombus material therein allowing retraction of the entire thrombus and/or one or more proximal layers to impact the thrombus material for removal via the aspiration catheter and/or proximal retraction of the thrombus removal device.

In some examples, the distal tip geometry of the mechanical engagement features may be configured to engage or otherwise impact thrombus material to provide manipulation or deformation (e.g., shearing, cutting, macerating, etc.) of the thrombus material therein. For example, referring to FIG. 16A, the distal tip geometry is generally a point or piercing tip, while FIG. 16G illustrates and example of a rounded or blunt tip. In some examples, the distal tip may be configured to impact the thrombus material. In some examples, a lateral edge or side of one or more mechanical engagement features may be configured to impact the thrombus material. In some examples, a combination of a tip and lateral edge may be configured to engage or impact the thrombus material. In some examples, a surface (e.g., distal and/or proximal surface) may be configured to impact the thrombus material. For example, a surface of one or more mechanical engagement features may be smooth, or irregular having teeth, knurled surface, or otherwise textured to provide increase engagement and manipulation of the thrombus material.

As described herein, one or more mechanical engagement features, layers, or arrays of mechanical engagement features may be actuated simultaneously, independently, selectively, etc. or a combination thereof. In some examples, any layer may be actuated based on the actuation or impact of one or more layers. For example, a proximal layer may be actuated, and one or more distal layers may be subsequently actuated once the proximal layer is actuated and engages the thrombus material. Some examples of layer or mechanical engagement feature articulation may include alternating mechanical engagement features (e.g., every-other mechanical engagement features) or any pattern of subsequent actuation. This manner of actuation or articulation of the mechanical engagement features may be configured to grab and/or pull thrombus material. Some examples of layer or mechanical engagement feature articulation may include sequential actuation of one or more mechanical engagement features or layers (e.g., mechanical engagement feature 1, mechanical engagement feature 2, mechanical engagement feature 3 . . . ). This manner of actuation may be configured to twist and/or rotate the thrombus material. Some examples of layer or mechanical engagement feature articulation may include varied axial position (e.g., offset or height of mechanical engagement features). This manner of actuation may be configured to retain, grab, pull, etc. the thrombus material. Some examples of layer or mechanical engagement feature articulation may include varied radial overlap of one or more mechanical engagement features of a layer. For example, mechanical engagement features may overlap, scissor, hook, curve, etc. or a combination thereof. This manner of actuation may be configured to pinch, cut, shear, etc. or a combination thereof the thrombus material. Some examples of layer or mechanical engagement features articulation may include iris closure (e.g., cyclonically, tangentially arrayed closure). This manner of actuation may be configured to twist and/or rotate the thrombus material.

In some implementations, the mechanical engagement features can serve to hold the clot within the distal end of the device, but not cut, macerate, or otherwise disrupt the clot. In one example, one or more mechanical engagement features can be actuated or positioned to hold the clot within the funnel, and the aspiration and/or jets may be oscillated on and off to break up and remote the clot from the patient. In some implementations, the jetting or fluid streams can be sequenced with mechanical engagement features actuation. For example, jetting can be turned on when the mechanical engagement features are actuated, and turned off when the mechanical engagement features are not actuated. In some examples, jetting can be turned on only after the mechanical engagement features are fully deployed or actuated, or alternatively, only when the mechanical engagement features are not deployed. Any combination of sequencing jetting and mechanical engagement features actuation is contemplated.

In some examples, actuation of one or more mechanical engagement features within the array may be based on manipulation or engagement of elongate members in operable communication with the mechanical engagement features. As described above, a sheath may be manipulated either axially or by rotation to actuate the mechanical engagement features. Movement of this sheath may be motorized or automated. In other examples, pull wires may be coupled to the mechanical engagement features and be configured to actuate the mechanical engagement features when the pull wires are engaged by a user or other actuation interface. In some examples, the pull wires can be attached or coupled to a motor configured to mechanically adjust a position of the pull wires to manipulate the mechanical engagement features. In some examples, mechanical engagement features actuation may be provided by a pneumatic system configured to adjust a pressure to the layers or mechanical engagement features for selective articulation of one or more mechanical engagement features, layers, or arrays. In some examples, operation of the mechanical engagement features may be facilitated by a thermal or electrical process. Actuation of the mechanical engagement features can be controlled, for example, with a user interface (e.g., button or GUI on a handle or console of the system). In some examples, a single user interface can be configured to control all mechanical engagement features at once. In other embodiments, multiple user interfaces or buttons can be configured to control the mechanical engagement features independently or in groups. For example, one or more mechanical engagement features may comprise a material or otherwise be configured to react to changes in temperature or electrical impulses transmitted to the mechanical engagement features. In some examples, one or more mechanical engagement features may be configured to automatically be actuated on contact (e.g., sufficient contact) with thrombus material within the distal end of the thrombus removal device. In some examples, actuation and articulation of one or more mechanical engagement features may be provided by engagement with a handle at a proximal end of the thrombus removal device (e.g., outside of a patient when in use). One or more engagement elements may be selectively controlled by a user to engage or otherwise actuate the mechanical engagement features and initiate their associated function. In some examples, one or more mechanical engagement features may be actuated by the sheath or delivery catheter. For example, a sheath may be advanced distally towards the mechanical engagement features causing actuation of the mechanical engagement features by pressure provided by the distal end of the sheath on the mechanical engagement features. In some examples, a proximal layer may be configured to transfer or transmit an actuation force to subsequent (e.g., distal layers).

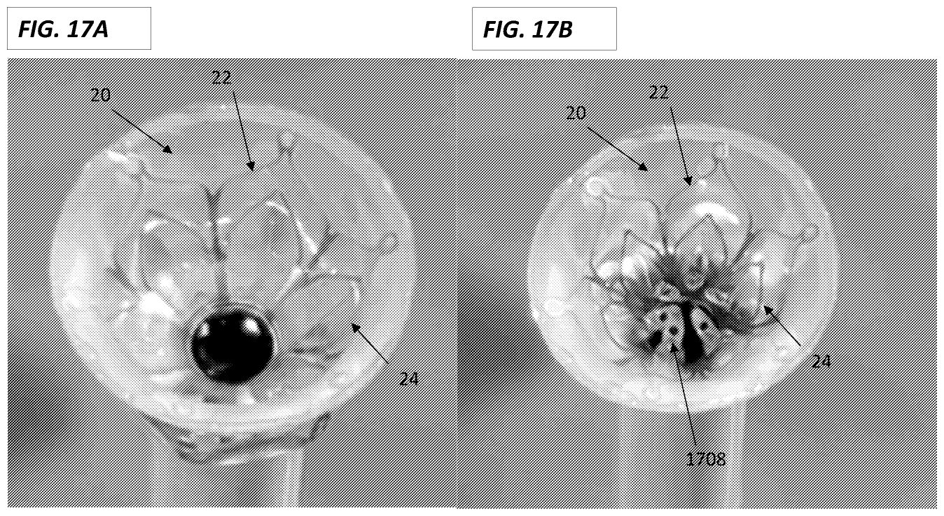

FIGS. 17A-17B illustrate an example of a thrombus removal device distal end including a funnel 20, funnel frame structure 22, mechanical engagement features frame structure 24, and mechanical engagement features 1708. In this embodiment, the funnel includes a compliant material that surrounds the funnel frame structure and at least a portion of the mechanical engagement features frame structure and mechanical engagement features. In some examples, the mechanical engagement features frame structure is at least partially covered or encapsulated by the compliant material, and the mechanical engagement features themselves are not covered by the compliant material. In some embodiments, the compliant material can comprise a polycarbonate-based thermoplastic urethane material such as Chronoflex.

In FIG. 17A, the mechanical engagement features are shown in an open configuration, in which the mechanical engagement features are expanded outwards and positioned adjacent to or abutting against the compliant material. In FIG. 17B, the mechanical engagement features are shown in a closed or actuated configuration, in which the mechanical engagement features are moved inwards from the funnel and optionally towards the aspiration lumen of the thrombus removal device. As with the other embodiments described herein, the mechanical engagement features can be actuated or controlled independently, in groups, all-together, or some combination thereof.

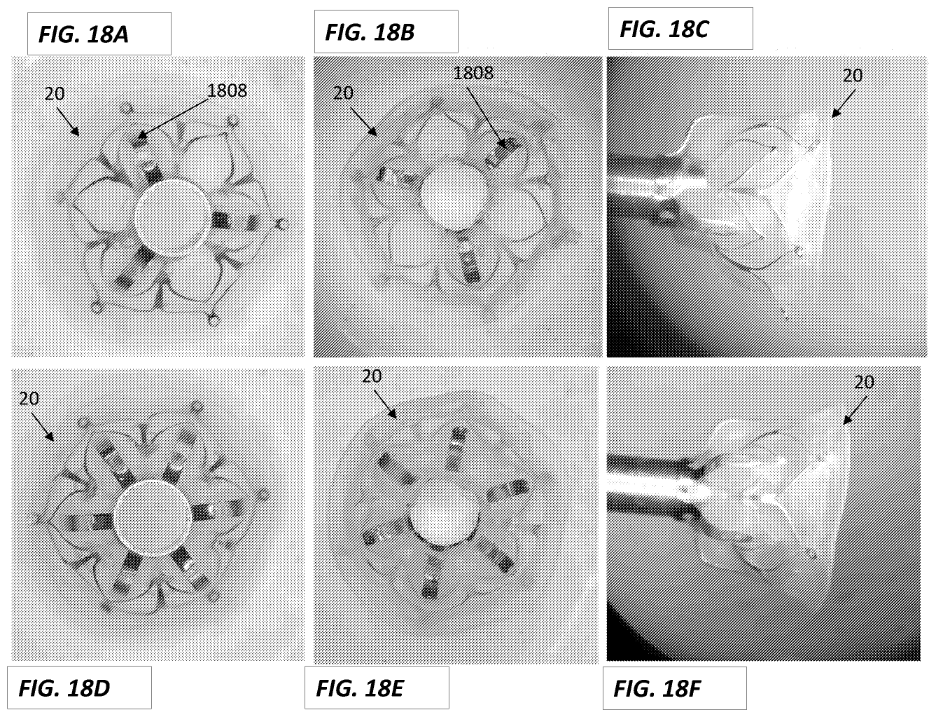

FIGS. 18A-18C illustrate an embodiment of a funnel that includes three mechanical engagement features 1808. FIG. 18A shows the mechanical engagement features in the open configuration, and FIG. 18B shows the mechanical engagement features in the closed or actuated configuration. FIG. 18C is a side-view of the funnel, including the funnel frame structure and mechanical engagement features frame structure embedded or surrounded by the compliant material. FIGS. 18D-18F illustrate similar views except with a six mechanical engagement feature design. It should be noted that, in both these embodiments, the mechanical engagement features are exposed or positioned outside of the compliant material in both the open and closed configurations. As with the other embodiments described herein, the mechanical engagement features can be actuated or controlled independently, in groups, all-together, or some combination thereof.

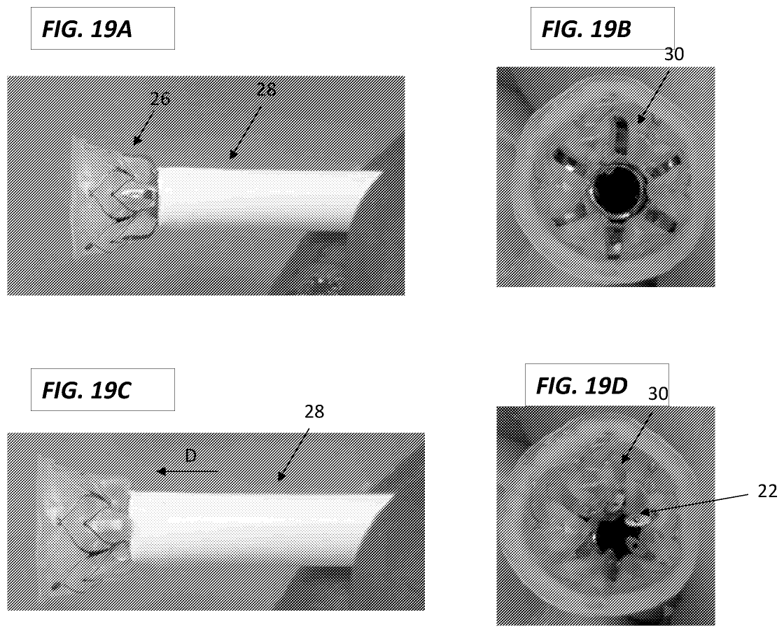

FIG. 19A is a side-view of a thrombus removal device including a sheath or delivery catheter 28. In this example, the sheath 28 is positioned proximally of the distal end and the mechanical engagement features frame structure 26, allowing the mechanical engagement features to assume their expanded position or configuration (e.g., allowing the mechanical engagement features to expand outwards to abut or rest against or within the compliant material of the funnel. FIG. 19B is a top view of this open configuration, with the mechanical engagement features resting inside optional “pockets” 30 within the compliant material. In the illustrated example, the pockets can have the same shape as the mechanical engagement features to allow the mechanical engagement features to be recessed from an interior of the funnel when in the expanded or open configuration. In one example, the pockets have a depth and shape that allows the mechanical engagement features to be flush with the interior surface of the funnel and compliant material.

FIG. 19C is another side-view of the thrombus removal device. In this example, the sheath 28 has been advanced distally relative to the thrombus removal device, causing the sheath to engage or contact the mechanical engagement features frame structure and push it distally. When the mechanical engagement features frame structure is advanced distally, it causes the mechanical engagement features themselves to assume the closed or actuated configuration. As with other embodiments described herein, the sheath can be moved either automatically (e.g., with a motor) or manually by the user.

FIG. 19D is a top view of this closed or actuated configuration, showing a view of the pockets 30 within the compliant material and the mechanical engagement features 22 pivoting or moving inwards towards the aspiration lumen of the device. As with the other embodiments described herein, the mechanical engagement features can be actuated or controlled independently, in groups, all-together, or some combination thereof.

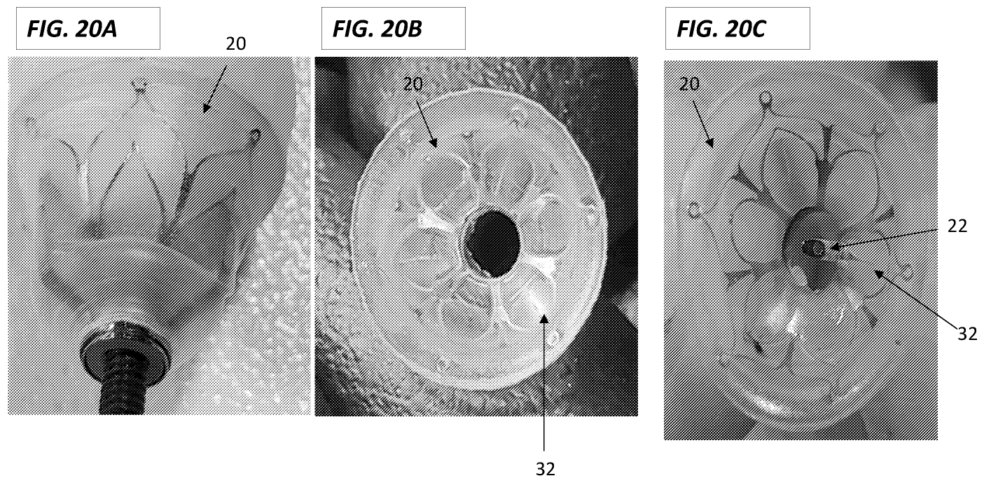

FIGS. 20A-20C illustrate an alternative embodiment, in which the mechanical engagement features are concealed by the compliant material when in the open configuration. FIG. 20A is a bottom view of the funnel 20, showing the mechanical engagement features frame structure and the funnel frame structure embedded in or covered by the compliant material. FIG. 20B shows the funnel including the mechanical engagement features in the open configuration, in which the mechanical engagement features themselves are recessed into pockets within the compliant material. In this example, the mechanical engagement features are configured to pass through slits 32 within the compliant material, to allow the mechanical engagement features to transition from the open configuration shown in FIG. 20B in which the mechanical engagement features are not visible and are covered by compliant material, and the closed or actuated configuration in FIG. 20C in which the mechanical engagement features 22 are able to pivot, move, or pass through slit(s) 32 to grab, cut, engage with, or manipulate a clot in the funnel. As with the other embodiments described herein, the mechanical engagement features can be actuated or controlled independently, in groups, all-together, or some combination thereof.

In some examples, any of the mechanical engagement features described herein may be positioned or locatable within the funnel frame structure or an expandable distal end or tip of a medical device, as described herein. In some examples, the mechanical engagement features may be positioned within the funnel frame structure until they are actuated to engage thrombus material therein. For example, a layer having any number of mechanical engagement features may be positioned within the compliant material of the funnel frame structure and once actuated may transition from within or on the funnel frame structure to engage the thrombus material.

### Assessing the Effectiveness/Completion of Treatment

Systems and methods are provided herein for assessing the effectiveness and/or completion progress of thrombectomy treatment. In some embodiments, the methods can be implemented entirely in software that resides on the thrombectomy device itself or is in communication with the device. In other embodiments, the methods can be implemented in combination with hardware disposed on or in the device that provides additional information to the system/device on treatment progress.

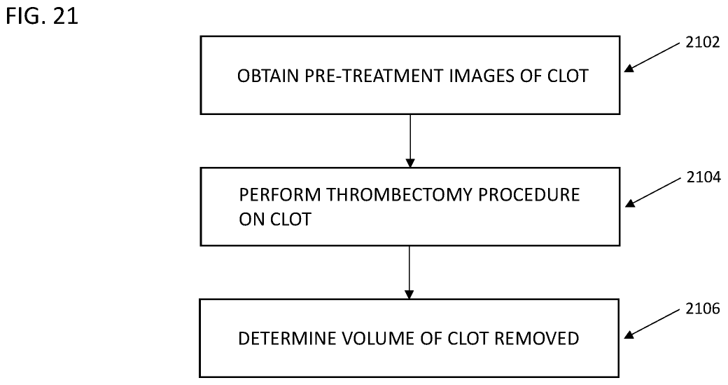

In one embodiment, a method of assessing the effectiveness or monitoring the progress of treatment can include assessing or determining the volume of clot removal based on pre-treatment imaging (e.g., CT). Referring to the flowchart of FIG. 21, the method can include, at step 2102, obtaining pre-treatment images of the clot to be removed or treated. In some embodiments, this can include obtaining CT images, ultrasound images, MRI images, or any other high resolution or high quality images of the target clot.

At step 2104, the method can then include performing a thrombectomy procedure on a targeted clot or clots using any of the devices and methods described herein.

Next, at step 2106, the method can include determining or calculating the volume of clot removed from the patient during the thrombectomy procedure. In some embodiments, this determination is done entirely in software, such as with algorithms that compare pre-treatment imaging to post-treatment imaging, determine the volume of pre-treatment clot to post-treatment clot, and identify the volume or percentage of clot removed.

In other embodiments, the determination can be based on sensor feedback from the thrombectomy device. For example, flow and/or pressure sensors outside the thrombectomy device or alternatively inside the aspiration lumen of the device can be used to measure or estimate the amount of clot removed in real-time. Alternatively, contrast agent can be delivered into the target region during treatment, such as with the jets or alternatively with a separate contrast agent lumen to allow for real-time imaging of the clot removal. In some embodiments, the contrast agent can be delivered from or near the funnel of the device. In some embodiments, additives can be added to the contrast agent which can adhere to the clot(s) and enhance the visibility of clots when the clots are removed under real-time imaging. This can then enable software or image processing solutions to estimate or determine the amount of clot removed during therapy.

In some embodiments, completion of the treatment can be determined or assessed based on a scoring system that is a composite of performance parameters (e.g., volume removed per step 2106 above) and/or physiological parameters (Sp02 increase/decrease, HR, respiratory rate, etc. recovering to normal ranges).

While the embodiments herein have been described as being intended to remove thrombi from a patient's vasculature, other applications of this technology are provided. For example, the devices described herein can be used for gastrointestinal procedures that include breaking up and removing hardened stool from the digestive tract of a patient, such as from the intestines or colon of a patient. In one embodiment, the device can be inserted into a colon or intestine of the patient (such as through the anus) and advanced to the site of hardened stool. Next, the aspiration system can be activated to engage the hardened stool with an engagement member (e.g., funnel) of the device. Finally, the jets or irrigation can be activated to break off pieces of the hardened stool and aspirate them into the system. Any of the techniques described above with respect to controlling the system or removing clots can be applied to the removal of hardened stool.

As one of skill in the art will appreciate from the disclosure herein, various components of the thrombus removal systems described above can be omitted without deviating from the scope of the present technology. As discussed previously, for example, the present technology can be used and/or modified to remove other types of emboli that may occlude a blood vessel, such as fat, tissue, or a foreign substance. Further, although some embodiments herein are described in the context of thrombus removal from a pulmonary artery, the disclosed technology may be applied to removal of thrombi and/or emboli from other portions of the vasculature (e.g., in neurovascular, coronary, or peripheral applications). Likewise, additional components not explicitly described above may be added to the thrombus removal systems without deviating from the scope of the present technology. Accordingly, the systems described herein are not limited to those configurations expressly identified, but rather encompasses variations and alterations of the described systems.

### CONCLUSION

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

A complete listing of the claims follows:

1. A thrombus removal device, comprising:
an elongate catheter having an aspiration lumen;
an aspiration source coupled to the aspiration lumen;
an expandable funnel coupled to the aspiration lumen and the elongate catheter;
at least one mechanical engagement feature disposed within the expandable funnel, wherein the at least one mechanical engagement feature is actuatable to move the at least one mechanical engagement feature within the expandable funnel.

2. The thrombus removal device of claim 1, wherein the at least one mechanical engagement feature is actuatable to move the at least one mechanical engagement within the expandable funnel towards the aspiration lumen.

3. The thrombus removal device of claim 1, wherein the at least one mechanical engagement feature is actuatable to move the at least one mechanical engagement feature generally radially within the expandable funnel across at least a portion of the expandable funnel.

4. The thrombus removal device of claim 1, wherein the at least one mechanical engagement feature comprises a cutting portion.

5. The thrombus removal device of claim 1, wherein the at least one mechanical engagement feature comprises a blunted tip.

6. The thrombus removal device of claim 1, further comprising:
a fluid source;
a fluid lumen positioned in the elongate catheter and in fluid communication with the fluid source;
a jet orifice positioned near or within the expandable funnel and in fluid communication with the fluid lumen, the jet orifice being configured to provide a fluid stream within the aspiration lumen or within the expandable funnel.

7. The thrombus removal device of claim 6, wherein the at least one mechanical engagement feature is actuatable to move the at least one mechanical engagement within the expandable funnel towards the fluid stream.

8. The thrombus removal device of claim 6, wherein at least one mechanical engagement feature is in fluid communication with the fluid lumen and is further configured to provide a second fluid stream within the aspiration lumen or within the expandable funnel.

9. The thrombus removal device of claim 1, wherein the expandable funnel comprises a funnel frame configured to self-expand the funnel to a fully expanded configuration.

10. The thrombus removal device of claim 9, wherein the expandable funnel further comprises a compliant material disposed over at least a portion of the funnel frame.

11. The thrombus removal device of claim 9, further comprising an actuatable frame mechanically coupled to the at least one mechanical engagement feature, the actuatable frame having at least a portion positioned proximally from the funnel frame.

12. The thrombus removal device of claim 11, further comprising a sheath configured to slide axially over the elongate catheter, wherein relative movement therebetween to place the sheath into contact with the actuatable frame moves the at least one mechanical engagement feature within the expandable funnel.

13. The thrombus removal device of claim 12, wherein placing the sheath into contact with the actuatable frame does not collapse or reduce a diameter of the expandable funnel.

14. The thrombus removal device of claim 9, wherein the at least one mechanical engagement feature is coupled to the funnel frame at a hinge.

15. The thrombus removal device of claim 14, wherein a distal portion of the at least one mechanical engagement feature extends from the hinge into the expandable funnel and a proximal portion of the at least one mechanical engagement feature extends outside of the funnel.

16. The thrombus removal device of claim 15, further comprising a sheath configured to slide axially over the elongate catheter, wherein relative movement therebetween to place the sheath into contact with the proximal portion of the at least one mechanical engagement feature moves the distal portion of the at least one mechanical engagement feature about the hinge within the expandable funnel.

17. The thrombus removal device of claim 15, further comprising a sheath configured to rotate around the elongate catheter, wherein rotating the sheath into contact with the proximal portion of the at least one mechanical engagement feature moves the distal portion of the at least one mechanical engagement feature about the hinge within the expandable funnel.

18. The thrombus removal device of claim 1, wherein the at least one mechanical engagement feature includes a pair of mechanical engagement features configured to collide at a pinch point when actuated.

19. The thrombus removal device of claim 1, wherein the at least one mechanical engagement feature includes a pair of mechanical engagement features configured to create a shearing action when actuated.

20. The thrombus removal device of claim 1, wherein the at least one mechanical engagement feature comprises a plurality of mechanical engagement features that are collectively actuated as a group.

21.-152. (canceled)
